(19) United States
(12) Patent Application Publication
(10) Pub. No.: US 2014/0371292 A1
(43) Pub. Date: Dec. 18, 2014

(54) Method for the controlled intracellular delivery of nucleic acids

(71) Applicants: Ahmed Besheer, Munich (DE); Matthaus Noga, Munich (DE); Gerhard Winter, Penzberg (DE); Ernst Wagner, Munich (DE); Daniel Edinger, Baldham (DE)

(72) Inventors: Ahmed Besheer, Munich (DE); Matthaus Noga, Munich (DE); Gerhard Winter, Penzberg (DE); Ernst Wagner, Munich (DE); Daniel Edinger, Baldham (DE)

(73) Assignee: Ludwig-Maximilians-Universitat Munchen, Munich (DE)

(21) Appl. No.: 14/125,914

(22) PCT Filed: Aug. 10, 2012
(86) PCT No.: PCT/EP2012/065735

§ 371 Date: Jul. 21, 2014

(30) Foreign Application Priority Data
Aug. 10, 2011 (EP) 11177175.4
Dec. 21, 2011 (EP) 11195030.9

(51) Int. Cl. A61K 47/36; A61K 31/713

(52) U.S. Cl. CPC A61K 47/36; A61K 31/713
USPC 514/44 A; 435/375

(57) ABSTRACT

The present invention relates to a method for the controlled intracellular delivery of nucleic acid molecules into one or more target cells, in particular tumor cells, the method comprising: providing a polymeric complex formed between one or more nucleic acid molecules to be delivered and one or more cationic carrier molecules, wherein at least a part of the one or more carrier molecules in the polymeric complex are covalently attached to hydroxyalkyl starch, and wherein the hydroxyalkyl starch is shielding the polymeric complex; allowing the shielded polymeric complex to get into contact with the one or more target cells; deshielding the polymeric complex by removing the hydroxyalkyl starch; and allowing the deshielded polymeric complex to internalize into the one or more target cells. Removal of the hydroxyalkyl starch can be accomplished enzymatically by exposing the polymeric complex to amylase. The invention also concerns the use of such method for the prevention and/or treatment of a condition selected from the group consisting of cancer, immune diseases, cardiovascular diseases, neuronal diseases, infections, and inflammatory diseases.

### FIELD OF THE INVENTION

The present invention relates to a method for the controlled delivery of nucleic acids to an intracellular target. Hydroxyalkyl starch is used for the effective shielding of the nucleic acid molecules during transport to the target cells, where the protective moiety is selectively removed, thus facilitating cellular uptake.

### BACKGROUND

The cell plasma membrane represents an efficient barrier that prevents most molecules that are not actively imported from cellular uptake. Consequently, the cell plasma membrane also hampers the targeted delivery of therapeutic substances. Only a small range of molecules having a particular molecular weight, polarity and/or net charge is able to (passively) diffuse through cell membranes. All other molecules have to be actively transported, e.g., by receptor-mediated endocytosis or via ATP-binding transporter molecules. In addition, molecules may also artificially be forced to pass the cell membrane (i.e. are transfected into cells), for example by means of electroporation, cationic lipids/liposomes, micro-injection, viral delivery or encapsulation in polymers. However, these methods are mainly utilized to deliver hydrophobic molecules and may have significant side effects, especially when employing viral gene delivery, thus preventing them from becoming an efficient tool for the controlled delivery of drugs or other therapeutically active agents into cells.

In particular, the requirement of targeted delivery has previously turned out to represent a major challenge in the development of RNAi (RNA interference)-based drugs. Such agents comprise small RNA molecules (e.g., siRNAs, miRNAs or shRNAs) that interfere with the expression of disease-causing or disease-promoting genes. Following the demonstration of RNAi in mammalian cells (Elbashir, S. M. et al. (2001) Nature 411, 494-498), it was quickly realized that this sequence-specific mechanism of posttranscriptional gene silencing might be harnessed to develop a new class of medicaments that could be applicable for the treatment of diseases which have not been accessible to therapeutic intervention so far (De Fougerolles, A. et al. (2007) Nat. Rev. Drug Discov. 6, 443-453). However, as RNAi takes place in the cytosol any RNA-based drugs have to pass the cell membrane in order to exert their therapeutic effect. Several methods have been described so far in order to accomplish this goal such as the use of lipids (Schroeder; A. et al. (2010) J. Intern. Med. 267, 9-21), viral carriers (Liu, Y. P: and Berkhout, B. (2009) Curr. Top. Med. Chem. 9, 1130-1143), polycationic nanoparticles (Howard, K. A. (2009) Adv. Drug Deliv. Rev. 61, 710-720), and cell penetrating peptides (Fonseca, S. B et al. (2009) Adv. Drug Deliv. Rev. 61, 953-964).

In addition, for the therapeutically effective treatment of a condition selective targeting of any nucleic acid-based drugs to the site of action is required in order to locally increase the drug concentration at the target (e.g., a solid tumor), reduce the extent of potentially adverse side effects (e.g., when employing cytotoxic drugs), and minimize the change for drug resistance.

Nucleic acids per se are poorly internalized by eukaryotic cells. This poor uptake is likely a defense mechanism to ensure the integrity of the cellular genome against entry by foreign DNA and RNA. Nucleic acid uptake is therefore challenged by the cells' natural tendency to repel foreign nucleic acid molecules, poor endosomal release, adverse immunogenic effects, and their instability and enzymatic degradability.

In order to overcome at least some of these obstacles carrier molecules, such as cationic lipids or polycations, are commonly used to condense the RNA or DNA molecules into polymeric complexes prior to application, thereby masking, at least to some extent, the nucleic molecules' polyanionic nature. However, in turn, such polymeric complexes usually having a positive surface charge tend to bind to anionic plasma proteins or erythrocytes in the bloodstream, thus leading to in vivo aggregation and accumulation in the lung, where they are rapidly eliminated by the mononuclear phagocyte system.

Accordingly, various attempts were made to shield these polymeric complexes during transport to the target site. One approach utilizes the hydrophilic polymer poly(ethylene glycol) (PEG) which is coupled to the polymeric nucleic acid complexes for masking the surface charges and decreasing the non-specific electrostatic interactions (Harris, J. M. et al. (2003) Nat. Rev. Drug Discov. 2, 214-221; Xu, L. et al. (2011) J. Pharm. Sci. 100, 38-52). PEGylation was in fact shown to prolong half-life and circulation time of the polymeric complexes in vivo.

However, on the other hand, the PEG coating reduces transfection efficiency in vitro and in vivo, an effect known as the “PEG-dilemma” (Mishra, S. et al. (2004) Eur. J. Cell Biol. 83, 97-111). It is speculated that PEGylation might interfere with endosomal esape of the complexes, cellular uptake as well as with nucleic acid release, thus resulting in poor transfection efficiencies.

In order to address this problem chemically modified PEG polymers including cleavable linker moieties were employed allowing the reversible shielding of the polymeric complexes (see, e.g., international patent publication WO 2011/026641; Walker, G. F. et al. (2005) Mol. Ther. 11, 418-425). Those modifications indeed resulted in some improvements in transfection efficiency but the process of deshielding of the polymeric complexes could not be effectively controlled such that the protective coat remains on the complexes during transport and is only removed at the final cellular target site.

Thus, it still remains an unresolved challenge how to transport and spatially concentrate nucleic acid-based drugs at the desired site of therapeutic intervention in order to reduce the overall drug concentration to be employed (i.e. administered to prevent or treat a medical condition) and thereby to minimize potentially adverse side effects.

Accordingly, there is a need for methods for the targeted delivery of therapeutic nucleic acid molecules that overcome the above-mentioned limitations. In particular, there is a need for such methods that enable intracellular delivery of nucleic acids into target cells with high transfection efficiency but without exerting significant cytotoxic and/or immunogenic effects during transport there.

Hence, it is an object of the present invention to provide such methods for the controlled intracellular delivery of nucleic acid molecules.

### SUMMARY OF THE INVENTION

In a first aspect, the present invention relates to a method for the controlled intracellular delivery of nucleic acid molecules into one or more target cells, the method comprising:
(a) providing a shielded polymeric complex formed between one or more nucleic acid molecules to be delivered and one or more cationic carrier molecules, wherein at least a part of the one or more carrier molecules in the polymeric complex are covalently attached to hydroxyalkyl starch, and wherein the hydroxyalkyl starch is shielding the polymeric complex;
(b) allowing the shielded polymeric complex to get into contact with the one or more target cells;
(c) deshielding the polymeric complex by removing the hydroxyalkyl starch; and
(d) allowing the deshielded polymeric complex to internalize into the one or more target cells.

In preferred embodiments, the hydroxyalkyl starch is hydroxyethyl starch. Particularly preferably, the hydroxyethyl starch has an average molecular weight in the range between 2 kDa and 300 kDa, and particularly in the range between 10 kDa and 200 kDa; and an average number of hydroxylethyl groups per glucose unit in the range between 0.1 and 2.0, and particularly in the range between 0.1 and 1.0.

In specific embodiments, in the shielded polymeric complex the molar ratio between free and hydroxyalkyl starch-modified carrier molecules is in the range between 1:99 and 99:1, and particularly in the range between 5:95 and 95:5.

In other preferred embodiments, the hydroxyalkyl starch is removed enzymatically by exposing the (shielded) polymeric complex to amylase, and particularly to α-amylase. The amylase may be exogenously added to the one or more target cells.

In further specific embodiments, the extent of modification with hydroxyalkyl starch is indicative of the amount of amylase required to substantially removing the hydroxyalkyl starch.

The shielded polymeric complex employed in the method may further comprise one or more targeting molecules for the specific delivery of the one or more nucleic acid molecules to the one or more target cells.

In preferred embodiments, the one or more carrier molecules are selected from the group consisting of cationic lipids, cationic cholesterol-complexes, cationic peptides, in particular poly-arginines and poly-lysines, polyalkylenimines, in particular polyethylenimine, protamines, and combinations thereof.

In other preferred embodiments, the one or more nucleic acid molecules to be delivered are selected from the group of RNA molecules, in particular siRNA molecules, miRNA molecules, and shRNA molecules, and precursor molecules thereof, and DNA molecules.

In further preferred embodiments, the one or more target cells are tumor cells. In particular embodiments, the tumor cells are amylase-producing tumor cells.

In a second aspect, the present invention relates to the use of a method as defined herein for the delivery of one or more therapeutically active nucleic acid molecules into one or more target cells. Preferably, the one or more therapeutically active nucleic acid molecules are applied for the prevention and/or treatment of a condition selected from the group consisting of cancer, immune diseases, cardiovascular diseases, neuronal diseases, infections, and inflammatory diseases.

Finally, in a third aspect, the present invention relates to a pharmaceutical composition, comprising a shielded polymeric complex as defined herein, and optionally further comprising amylase, for use in the prevention and/or treatment of a condition selected from the group consisting of cancer, immune diseases, cardiovascular diseases, neuronal diseases, infections, and inflammatory diseases.

Other embodiments of the present invention will become apparent from the detailed description hereinafter.

### DESCRIPTION OF THE DRAWINGS

FIG. 1: Enzymatic degradation of hydroxyethyl starch with α-amylase

Solutions of hydroxyethyl starch (HES) having an average molecular weight of 70 kDa (“HES70”) and 20 kDa (“HES20”) were each prepared at a concentration of 5 mg/ml in HEPES buffered glucose, pH 6.0 (“HBG”) and phosphate buffered saline, pH 7.4 (“PBS”), respectively. Pancreatic α-amylase (“AA”) was added at a final concentration of 40 U/I. Degradation of HES70 and HES20 was monitored at 25° C. (A and B), and 37° C. (C and D), respectively. Samples were taken after incubation for 0, 0.5, 1, 2, 4, 6, and 24 h and heated to 99° C. for 3 min to stop enzyme activity. The reduction in the molar mass of HES was analyzed by means of asymmetric flow field flow fractionation using the Wyatt Eclipse 2 AF4 system (Wyatt Technology Corp., Santa Barbara, Calif., USA). Panel (E) depicts the effect of the pH value (6.0 versus 7.1) on the degradation of HES20.

FIG. 2: Preparation of HES-PEI conjugates

Synthesis scheme for the direct coupling of polyethylenimine (PEI) to hydroxyethyl starch (HES) is accomplished via the formation of a Schiff's base and subsequent reductive amination (adapted from Wollrab, A. (ed.) (2009) Organische Chemie, in: Springer Verlag Berlin, Heidelberg, p. 485-496).

FIG. 3: Characterization of HES-PEI conjugates

Conjugates of HES20 and HES70 with PEI22 (i.e. polyethylenimine having an average molecular weight of 22 kDa) were prepared in a molar ratio HES:PEI of 25:1 as described (Kircheis R. et al. (2001) Gene Ther. 8, 28-40) and characterized with respect to coupling efficiency and molar ratio by nuclear magnetic resonance spectroscopy. For the ¹H-NMR measurements, 10 mg samples of HES20-PEI22 (top) and HES70-PEI22 (bottom) were dissolved in D₂O, and spectra (with peak assignment) were obtained by using a JNMR-GX500 (500 MHz) spectrometer (Jeol GmbH, Eching, Germany).

FIG. 4: Characterization of HES-PEI conjugates

The HES-PEI conjugates were further characterized by a combination of size exclusion chromatography (SEC) and multi-angle light scattering (MALS). Samples of HES20-PEI22 (top) and HES70-PEI22 (bottom) were prepared at a concentration of 5 mg/ml. Control samples (mixture of HES and PEI as well as free PEI) were prepared at a concentration of 1 mg/ml. SEC was performed by using a TSKgel G5000PWXL-CP column (7.8 mm×30.0 cm; Tosoh Bioscience GmbH, Stuttgart, Germany) at a flow rate of 0.5 ml/min. MALS was performed at 18 angles using the Eclipse 2 separation system (Wyatt Technology Corp. Santa Barbara, Calif., USA) and the 1100 Series Agilent HPLC system (Agilent Technologies, Palo Alto, Calif., USA). Chromatograms were prepared by means of the ASTRA software package (Wyatt Technology Corp).

FIG. 5: Characterization of polymeric nucleic acid complexes

Polymeric complexes (“naked Px”) were prepared via the rapid addition of PEI to pCMVLuc plasmid DNA (pDNA) (final DNA concentration of 20 μg/ml in HBG, pH 7.1) at N/P ratios of 3.6, 4.8, 6.0, 7.2, and 8.0 (i.e. the molar ratio of PEI nitrogen atoms to pDNA phosphate atoms), and then incubated at room temperature (RT) for 30 minutes prior to analysis. HESylated polymeric complexes were produced by partially replacing PEI with HES-modified PEI. HES70-PEI/DNA (“HES70Px”) and HES20-PEI/DNA (“HES20Px”) complexes were each generated with PEI:HES-PEI ratios of 95:5, 90:10, and 85:15, respectively. PEG20-PEI complexes (“PEG20Px”) were prepared with a PEI:PEG-PEI ratio of 90:10. Particle size and surface charge (zeta potential) determinations of the various polyplexes were performed in HBG, pH 6.0 or pH 7.1 using a Malvern Zetasizer Nano ZS (Malvern Instruments, Worcestershire, United Kingdom). Data represent means of at least three experiments. The upper panel shows the particle size distribution of different polymeric complexes as a function of increasing N/P ratios. The middle panel represents an enlarged illustration of the smaller particle size region of the upper panel. The bottom panel depicts the zeta potential distribution of different polymeric complexes as a function of increasing N/P ratios.

FIG. 6: Characterization of polymeric nucleic acid complexes

In order to analysis the stability of the polymeric complexes, the particle size (“SIZE”) and zeta potential (“ZP”) distributions of PEI complexes (“nPx”; A) and HES70-PEI complexes (“70Px”; B) in HBG buffer with pH 6 and pH 7.1 were determined as a function of time using the same experimental approach as in FIG. 5.

FIG. 7: Effect of α-amylase on polymeric DNA complexes

HES70-PEI complexes with an N/P ratio of 6.0, and PEI:HES-PEI ratios of 95:5, 90:10, and 85:15, respectively, were prepared as described in FIG. 5 and incubated for 30 min at room temperature. The HES was removed from the complexes by adding α-amylase (“AA”) in a final concentration of 40 U/I, and the samples were incubated at 37° C. and pH 6.0. The particle size (“SIZE”) and zeta potential (“ZP”) distributions (top) of the complexes were determined as a function of time using the same experimental approach as in FIG. 5. Bottom graphs show the ZP alone. Data represent means of at least three experiments.

FIG. 8: QCM-D experiments

Enzymatic degradation of HES30-PEI conjugates (A&C), HES60-PEI conjugates (B&D) and HES70-PEI (E) was analyzed as a function of time in the presence of α-amylase (“AA”) having an activity of 100 U/I and 300 U/I, respectively. Quartz crystal microbalance with dissipation (QCM-D) was performed on a Q-Sense E4 instrument (Q-Sense, Gothenburg, Sweden). One entire QCM-D run included the following five sections: (1) rinsing of the system with buffer (15 min); (2) polymer adsorption onto the SiO₂ sensor (5 min sample flow, 10 min without flow); (3) rinsing of the system with buffer (15 min); (4) start of enzymatic degradation by addition of α-amylase (5 min sample flow, 55 min without flow); and (5) rinsing of the system with buffer (15 min).

FIG. 9: Erythrocyte aggregation assay

A suspension of erythrocytes (2% (v/v) in PBS, pH 7.4) from 3 months old male C57BL/6 mice were mixed with HES70Px or HES20Px complexes in HBG pH 7.1 (as described in FIG. 5: final concentration of 1 μg pDNA, N/P ratio of 6.0, and PEI:HES-PEI ratios of 95:5, 90:10, and 85:15, respectively). α-amylase (“AA”) was added in a final concentration of 40 U/I. Buffer, buffer+AA, naked PEI-DNA complexes (“nPx”) and PEG20Px (both as in FIG. 5) were used as controls. The solutions were incubated in 24-well plates (Corning Costar, Sigma-Aldrich, Steinheim, Germany) for 90 min at 37° C. under constant gentle agitation. For microscopic analysis, pictures were taken with a Keyence VHX-500F digital microscope (Keyence Corporation, Osaka, Japan) with a 1000-fold magnification.

FIG. 10: Luciferase gene expression analysis

In vitro pDNA transfection efficiency was evaluated in murine N2A neuroblastoma (A) and human HUH7 hepatoma cell lines (B). The polymeric complexes described in FIG. 5 (final concentration of 200 ng pDNA/well) were added to 1×10⁴ cells in 100 μl medium in the presence or absence of 40 U/I α-amylase. 24 h after pDNA transfection, the cells were lyzed and the luciferase activity determined (Luciferase Assay System, Promega, Mannheim, Germany; Centro LB 960 luminometer, Berthold, Bad Wildbad, Germany). Metabolic activity of the transfected N2A (C) and HUH7 (D) cells was analyzed by means of a MTT assay (Sigma-Aldrich, Steinheim, Germany). The formazan reaction product quantified by a plate reader (Tecan, Groedig, Austria) at 590 nm with background correction at 630 nm and expressed as % of control. Data represent means of at least three experiments.

FIG. 11: Luciferase gene expression analysis

In vitro pDNA transfection efficiency was evaluated in murine N2A neuroblastoma cells by determining luciferase reporter gene expression (A, B) and metabolic activity (viability; C, D) as described in FIG. 10. Surface charge shielding (A, C) and deshielding (B, D) studies were performed in cell culture medium in the presence or absence of α-amylase. Analysis of the reporter gene expression and the corresponding metabolic activity was carried out 24 h after treatment with the polymeric complexes. Numbers at the x-axis represent the percentage of HESPEI to PEI, whereas the numbers in square brackets represent the degree of molar substitution of HES.

FIG. 12: Effect of α-amylase activity

HES70-PEI complexes with an N/P ratio of 6.0, and PEI:HES-PEI ratios of 90:10 were prepared as in FIG. 5. The HES was removed from the complexes by adding α-amylase (“AA”) in a final concentration of 40 U/I and 100 U/I, respectively. The samples were incubated at 37° C. and pH 6.0. The zeta potential distribution of the complexes was determined as a function of time (A). Luciferase gene expression in N2A neuroblastoma cells was determined in the presence or absence of 100 U/I AA as described in FIG. 9. Naked PEI-DNA complexes (“LPEI22”) and PEG20-PEI complexes (“PEG20”; having a PEI:PEG-PEI ratio of 90:10) were used as controls (B). Data represent means of at least three experiments.

FIG. 13: In vivo luciferase expression in lung and tumor tissues of N2A tumor-bearing NJ mice

Polymeric complexes were prepared by the respective rapid addition of PEI and HES70-PEI to pCMVLuc plasmid DNA (pDNA; final DNA concentration of 200 μg/ml in HGB, pH 7.1, N/P ratio 6.0), and incubated prior to analysis for 30 min at room temperature). An amount of 250 μl solution of polymeric complexes was injected into the tail vein of NJ mice. Naked LPEI and PEGylated particles (30% PEG20-PEI) served as controls. Luciferase expression in lung tissue (left) and tumor tissue (right) is presented as relative light units (RLU) per mg organ (A, B) or RLU per organ (C, D) (n=4 mice per group).

FIG. 14: Gene expression in lung and tumor tissues of N2A-tumor-bearing A/J mice after systemic administration of polymeric complexes

The experimental approach was the same as in FIG. 13. The incorporation of the non-biodegradable PEG-PEI and HES60-PEI[1.3] into the LPEI-based polymeric complexes strongly reduced, by shielding of the polymeric complexes, the gene expression in lung tissue (left) and tumor tissue (right) by up to 2-4 orders of magnitude. The shielding and deshielding of biodegradable HES70-coatings resulted in safe particles with maintenance of the initial tumor transfection efficiency. Naked LPEI and PEGylated particles served as controls. The luciferase expression is presented as relative light units (RLU) per mg organ (A, B) and RLU per organ (C, D) (n=5 mice per group).

FIG. 15: Binding and uptake capacity of the polyplexes using flow cytometry

The uptake of HES20Px and HES70Px (each with PEI:HES-PEI ratios of 90:10; prepared as described in FIG. 5) was studied in N2A neuroblastoma cells in the presence or absence of 40 U/I α-amylase (“AA”). 50 μl of the respective polyplexes (20 μg/ml pDNA, 10% of which is Cy5-labeled) were added to aliquots of 1×10⁵ cells. For binding studies (A and B), the treated cells were kept at 4° C. for 30 min, washed with PBS (phosphate buffered saline), and trypsinized (Trypsin/EDTA solution, Biochrome AG, Berlin, Germany). Samples used for the determination of cellular uptake (C and D) were incubated at 37° C. for 60 min. Afterwards, the polyplexes were disassembled by adding 1000 I.E./ml heparin, and the cells trypsinized. The percentage of Cy5 positive cells (A and C) was determined by measuring the excitation of Cy5 at 635 nm. The mean fluorescence intensity (“MFI”, B and D) was determined by measuring the emission of Cy5 at 665 nm.

### DETAILED DESCRIPTION OF THE INVENTION

The present invention is based on the unexpected finding that the use of hydroxyalkyl starch for the shielding of polymeric nucleic acid complexes enabled a novel experimental approach for the efficient (non-viral) intracellular delivery of therapeutic nucleic acids. Removal of the protective hydroxyalkyl starch from the complexes is achieved enzymatically by amylases wherein the rate and extent of degradation can be manipulated via the degree of molar substitution (i.e. hydroxyalkylation) of the hydroxyalkyl starch. This method allows for a precise control of the time point when the protective coat is removed, in particular degradation can be deferred until the polymeric complexes have been targeted to the desired cellular site of action. This method should also allow engineering of “customized” polymeric nucleic acid complexes having a specific degradation profile for the controlled intracellular delivery to particular target cells, e.g., adapted to a given medical condition to be treated.

The present invention illustratively described in the following may suitably be practiced in the absence of any element or elements, limitation or limitations, not specifically disclosed herein.

Where the term “comprising” is used in the description and the claims, it does not exclude other elements or steps. For the purposes of the present invention, the term “consisting of” is considered to be a preferred embodiment of the term “comprising”. If hereinafter a group is defined to comprise at least a certain number of embodiments, this is also to be understood to disclose a group, which preferably consists only of these embodiments.

Where an indefinite or definite article is used when referring to a singular noun, e.g., “a”, “an” or “the”, this includes a plural of that noun unless specifically stated otherwise.

In case, numerical values are indicated in the context of the present invention the skilled person will understand that the technical effect of the feature in question is ensured within an interval of accuracy, which typically encompasses a deviation of the numerical value given of ±10%, and preferably of ±5%.

Furthermore, the terms first, second, third, (a), (b), (c), and the like, in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Further definitions of term will be given in the following in the context of which the terms are used. The following terms or definitions are provided solely to aid in the understanding of the invention. These definitions should not be construed to have a scope less than understood by a person of ordinary skill in the art.

In a first aspect, the present invention relates to a method for the controlled intracellular delivery of nucleic acid molecules into one or more target cells, the method comprising:
(a) providing a shielded polymeric complex formed between one or more nucleic acid molecules to be delivered and one or more cationic carrier molecules, wherein at least a part of the one or more carrier molecules in the polymeric complex are covalently attached to hydroxyalkyl starch, and wherein the hydroxyalkyl starch is shielding the polymeric complex;
(b) allowing the shielded polymeric complex to get into contact with the one or more target cells;
(c) deshielding the polymeric complex by removing the hydroxyalkyl starch; and
(d) allowing the deshielded polymeric complex to internalize into the one or more target cells.

The term “intracellular delivery”, as used herein, is to be understood in a broad sense including both the transport of nucleic acid molecules to and their transfection in given target cells. In other words, the term refers to the artificially forced passage (“internalization”) of nucleic acid molecules through the plasma membrane of target cells. The directed transport of the nucleic acid molecules to particular cells is herein also referred to as “targeting”. In specific embodiments, at least 0.05%, at least 0.1%, at least 0.5%, at least 1%, at least 2%, at least 5%, at least 10% or at least 20% of the one or more nucleic acid molecules initially employed for performing the method are internalized in the one or more target cells.

The term “nucleic acid molecule”, as used herein, denotes any nucleic acid molecules including naturally occurring nucleic acids such as deoxyribonucleic acid (DNA) or ribonucleic acid (RNA) as well as artificially designed nucleic acids that are chemically synthesized or generated by means of recombinant gene technology including, e.g., nucleic acid analogs such as inter alia peptide nucleic acids (PNA) or locked nucleic acids (LNA), (see, e.g., Sambrook, J., and Russel, D. W. (2001), Molecular cloning: A laboratory manual (3rd Ed.) Cold Spring Harbor, N.Y., Cold Spring Harbor Laboratory Press). Specific examples of naturally occurring nucleic acids include DNA sequences such as genomic DNA or cDNA molecules as well as RNA sequences such as hnRNA, mRNA or rRNA molecules or the reverse complement nucleic acid sequences thereof. Such nucleic acids can be of any length and can be either single-stranded or double-stranded molecules. Typically, the nucleic acids to be employed herein are 10 to 10.000 nucleotides in length, e.g., 15 to 7.000 nucleotides, 20 to 5.000 nucleotides, 25 to 3.000 nucleotides, 30 to 2.000 nucleotides or 35 to 1.000 nucleotides. However, in some embodiments, nucleic acids having a length of more than 10.000 nucleotides may be employed. In preferred embodiments, the nucleic acids are 10 to 1.000 nucleotides in length, e.g., 15 to 800 nucleotides, 15 to 600 nucleotides, 20 to 600 nucleotides, 20 to 400 nucleotides, 25 to 400 nucleotides or 25 to 200 nucleotides. The term “nucleotide” is again to be understood as referring to both ribonucleotides and deoxyribonucleotides (i.e. RNA and DNA molecules).

In specific embodiments, the nucleic acid molecules employed herein are RNA molecules, and particularly small non-coding RNA molecules (i.e. RNAs not translated into a peptide or protein such as snRNAs, snoRNAs, stRNAs, siRNAs, miRNAs, and shRNAs). Preferably, the RNA molecules are selected from the group consisting of siRNA molecules, miRNA molecules, shRNA molecules, and precursor molecules thereof.

The term “miRNA molecule” (or “miRNA”), as used herein, is given its ordinary meaning in the art (reviewed, e.g. in Bartel, D. P. (2004) Cell 23, 281-292; He, L. and Hannon, G. J. (2004) Nat. Rev. Genet. 5, 522-531). Accordingly, the term “microRNA” denotes an endogenous RNA molecule derived from a genomic locus that is processed from transcripts that can form local RNA precursor miRNA structures. The mature miRNA is usually 20, 21, 22, 23, 24, or 25 nucleotides in length, although other numbers of nucleotides may be present as well, for example 18, 19, 26 or 27 nucleotides.

The miRNA encoding sequence has the potential to pair with flanking genomic sequences, placing the mature miRNA within an imperfect RNA duplex (herein also referred to as stem-loop or hairpin structure or as pre-miRNA), which serves as an intermediate for miRNA processing from a longer precursor transcript. This processing typically occurs through the consecutive action of two specific endonucleases termed Drosha and Dicer, respectively. Drosha generates from the primary transcript (referred to as “pri-miRNA”) a miRNA precursor (herein also denoted “pre-miRNA”) that typically folds into a hairpin or stem-loop structure. From this miRNA precursor a miRNA duplex is excised by means of Dicer that comprises the mature miRNA at one arm of the hairpin or stem-loop structure and a similar-sized segment (commonly referred to miRNA*) at the other arm. The miRNA is then guided to its target mRNA to exert its function, whereas the miRNA* is degraded in most cases. Depending on the degree of complementarity between the miRNA and its target, miRNAs can guide different regulatory processes. Target mRNAs that are highly complementary to miRNAs are specifically cleaved by mechanisms identical to RNA interference (RNAi) and the miRNAs function as short interfering RNAs (siRNAs). Target mRNAs with less complementarity to miRNAs are either directed to cellular degradation pathways and/or are translationally repressed. However, the mechanism of how miRNAs repress translation of their target mRNAs is still a matter of controversy.

In some embodiments, the one or more nucleic acid molecules attached to the at least one peptide molecule as defined herein are mature miRNA molecules. In other embodiments, miRNA precursor molecules are employed. The term “miRNA precursor” (or “pre-miRNA”), as used herein, refers to the portion of a miRNA primary transcript from which the mature miRNA is processed. Typically, the pre-miRNA folds into a stable hairpin (i.e. a duplex) or a stem-loop structure. The hairpin structures range from 50 to 120 nucleotides in length, typically from 55 to 100 nucleotides, and preferably from 60 to 90 nucleotides (counting the nucleotide residues pairing to the miRNA (i.e. the “stem”) and any intervening segment(s) (i.e. the “loop”) but excluding more distal sequences).

The term “siRNA molecule” (or “siRNA”), as used herein, is also given its ordinary meaning in the art (reviewed, e.g., in Dorsett, Y. and Tuschl, T. (2004) Nat. Rev. Drug Disc. 3, 318-329; Rana, T. M. (2007) Nat. Rev. Mol. Cell Biol. 8, 23-36). Accordingly, a “siNA” denotes a double-stranded RNA molecule, typically having 2 nucleotides overhang at their 3′-ends and phosphate groups at their 5′-ends. A mature siRNA is usually 20, 21, 22, 23, 24, or 25 nucleotides in length, although other numbers of nucleotides may be present as well, for example 18, 19, 26 or 27 nucleotides. Within the present invention, siRNA precursor molecules having a length of up to 49 nucleotides may be employed as well. The mature siRNA is processed from such precursor by Dicer.

Traditionally, the term “siRNA” was used to refer to interfering RNAs that are exogenously introduced into cells. In the meantime, endogenous siRNAs have been discovered in various organisms and fall into at least four classes: trans-acting siRNAs (tasiRNAs), repeat-associated siRNAs (rasiRNAs), small-scan (scn)RNAs and Piwi-interacting (pi)RNAs (reviewed, e.g., in Rana, T. M. (2007) supra).

One strand of the siRNA is incorporated into the RNA-induced silencing complex (RISC). RISC uses this siRNA strand to identify mRNA target molecules that are at least partially complementary to the incorporated siRNA strand, and then cleaves these target mRNAs. The siRNA strand that is incorporated into RISC is known as the guide strand or the antisense strand. The other siRNA strand, known as the passenger strand or the sense strand, is eliminated from the siRNA and is at least partially homologous to the target mRNA. Those of skill in the art will recognize that, in principle, either strand of a siRNA can be incorporated into RISC and function as a guide strand. However, siRNA design (e.g., decreased siRNA duplex stability at the 5′ end of the desired guide strand) can favor incorporation of the desired guide strand into RISC. The antisense strand of a siRNA is the active guiding agent of the siRNA in that the antisense strand is incorporated into RISC, thus allowing RISC to identify target mRNAs with at least partial complementarity to the antisense siRNA strand for cleavage or translational repression. RISC-mediated cleavage of mRNAs having a sequence at least partially complementary to the guide strand leads to a decrease in the steady state level of that mRNA and of the corresponding protein.

The term “shRNA molecule” (i.e. short hairpin RNA molecule), as used herein, denotes an artificial single-stranded interfering RNA molecule comprising both sense and anti-sense strand of a “siRNA duplex” in a stem-loop or hairpin structure. The stem of this hairpin structure typically ranges from 19 to 29 nucleotides in length, and a loop typically ranges from 4 to 15 nucleotides in length (see, e.g., Siolas, D. et al. (2004) Nat. Biotechnol. 23, 227-231). Usually, an shRNA molecule is encoded within a DNA expression vector under the control of a RNA polymerase III promoter (e.g., the U6 promoter).

In some embodiments, the RNA molecules described above have a backbone structure exclusively comprising ribonucleotide units. In other embodiments, such a RNA molecule comprises at least one ribonucleotide backbone unit and at least one deoxyribonucleotide backbone unit. Furthermore, the RNA molecule may contain one or more modifications of the ribose 2′-OH group into a 2′-O-methyl group or 2′-O-methoxyethyl group (also referred to as “2′-O-methylation”), which prevented nuclease degradation and also endonucleolytic cleavage by the RNA-induced silencing complex nuclease, leading to irreversible inhibition of the small RNA molecule. Another possible modification, which is functionally equivalent to 2′-O-methylation, involves locked nucleic acids (LNAs) representing nucleic acid analogs containing one or more LNA nucleotide monomers with a bicyclic furanose unit locked in an RNA-mimicking sugar conformation (cf., e.g., Orom, U. A. et al. (2006) Gene 372, 137-141). In other embodiments, the nucleic acid molecules employed herein represent silencers of endogenous miRNA expression. One example of such silencers are chemically engineered oligonucleotides, named “antagomirs”, which represent single-stranded 21-23 nucleotide RNA molecules conjugated to cholesterol (Krutzfeldt, J. et al. (2005) Nature 438, 685-689). Alternative microRNA inhibitors that can be expressed in cells as RNAs produced from transgenes are termed “microRNA sponges.” These competitive inhibitors are transcripts expressed from strong promoters, and containing multiple tandem-binding sites to a microRNA of interest (Ebert, M. S. et al. (2007) Nat. Methods 4, 721-726).

In further specific embodiments, the nucleic acid molecules are DNA molecules, including inter alia aptamers (also known as “DNA decoy drugs”). Aptamers bind to a target protein and thereby interfere with its function. Typically, aptamers are single-stranded or double-stranded oligonucleotides having a length of 10 to 80 nucleotides, e.g. 15 to 60 nucleotides, 18 to 50 nucleotides or 20 to 40 nucleotides. Other DNA molecules to be employed herein are DNA-based vaccines which are used to elicit an immune response (see, e.g., Irvine, A. S. et al. (2000) Nat. Biotech. 18, 1273-1278).

The term “one or more” nucleic acid molecules, as used herein, does not only refer to the total number of nucleic acid molecules employed but also to the types of nucleic acid molecules. For example, the method may be performed using one or more miRNA molecules (having the same or different nucleotide sequences), or one or more DNA aptamers (having the same or different nucleotide sequences), or a combination of at least one miRNA molecule and at least one DNA aptamer.

A nucleic acid molecule employed in the present invention may be present as an integral part of a genetic construct (also commonly denoted as an “expression cassette”) that enables its expression. A genetic construct is referred to as “capable of expressing a nucleic acid molecule” or capable “to allow expression of a nucleic acid (i.e. nucleotide) sequence” if it comprises sequence elements which contain information regarding to transcriptional and/or translational regulation, and if such sequences are “operably linked” to the nucleotide sequence encoding the peptide.

The precise nature of the regulatory regions necessary for gene expression may vary among species, but in general these regions comprise a promoter which, in prokaryotes, contains both the promoter per se, i.e. DNA elements directing the initiation of transcription, as well as DNA elements which, when transcribed into RNA, will signal the initiation of translation. Such promoter regions normally include 5′ non-coding sequences involved in initiation of transcription and translation, such as the −35/−10 boxes and the Shine-Dalgarno element in prokaryotes or the TATA box, CAAT sequences, and 5′-capping elements in eukaryotes. These regions can also include enhancer or repressor elements as well as translated signal and leader sequences for targeting the native polypeptide to a specific compartment of a host cell. Suitable prokaryotic promoters include inter alia the lacUV5, tet and tac promoters of E. coli and the T3, T7, and SP6 phage promoters. Suitable eukaryotic promoters include inter alia the SV40 early and late promoters, the RSV and CMV promoters. The 3′ non-coding sequences may contain regulatory elements involved in transcriptional termination, polyadenylation, and the like. If, however, these termination sequences are not satisfactory functional in a particular host cell, then they may be substituted with signals functional in that cell. The skilled person is well aware of all these regulatory elements, and how to select such elements suitable for the expression of a nucleic acid molecule in a given setting.

The nucleic acid molecules employed in the invention, optionally as part of an expression cassette, may also be comprised in a vector or other cloning vehicle, e.g., a plasmid, cosmid, phagemid, virus, bacteriophage, artificial chromosome, or another vehicle commonly used in genetic engineering. The vector may be an expression vector that is capable of directing the expression of the nucleic acid molecule of the invention. Large numbers of suitable vectors are commercially available. The skilled person is well aware how to determine which vectors are suitable for expressing a nucleic acid molecule of interest in a given setting.

The term “target cell”, as used herein, refers to any cell susceptible to the delivery (including transfection) of nucleic acid molecules. The term “one or more”, as used herein, is to be understood not only to include individual cells but also tissues, organs, and organisms.

In specific embodiments, the method is performed as an in vitro method.

The one or more target cells may be part of a sample derived from a subject, typically a mammal such as a mouse, rat, hamster, rabbit, cat, dog, pig, cow, horse or monkey, and preferably a human. Such samples may include body tissues (e.g., biopsies or resections) and body fluids, such as blood, sputum, and cerebrospinal fluid. The samples may contain a single cell, a cell population (i.e. two or more cells) or a cell extract derived from a body tissue, and may be used in unpurified form or subjected to any enrichment or purification step(s) prior to use. The skilled person is well aware of various such purification methods (see, e.g., Sambrook, J., and Russel, D. W. (2001), supra; Ausubel, F. M. et al. (2001) Current Protocols in Molecular Biology, Wiley & Sons, Hoboken, N.J., USA). In specific embodiments, the sample is a blood sample such as whole blood, plasma, and serum. The term “whole blood”, as used herein, refers to blood with all its constituents (i.e. both blood cells and plasma). The term “plasma”, as used herein, denotes the blood's liquid medium. The term “serum”, as used herein, refers to plasma from which the clotting proteins have been removed. In other specific embodiments, the sample is re-administered to the subject from whom it was derived once the method of the invention has been performed thereon. For example, a blood sample comprising “transfected” target cells containing the one or more nucleic acid molecules to be delivered may be re-injected into the subject, e.g. intravenously.

In preferred embodiments, the one or more target cells are tumor cells. The term “tumor” (also referred to as “cancer”), as used herein, generally denotes any type of malignant neoplasm, that is, any morphological and/or physiological alterations (based on genetic re-programming) of target cells exhibiting or having a predisposition to develop characteristics of cancer as compared to unaffected (healthy) control cells. Examples of such alterations may relate inter alia to cell size and shape (enlargement or reduction), cell proliferation (increase in cell number), cell differentiation (change in physiological state), apoptosis (programmed cell death) or cell survival. Exemplary tumor cells include inter alia those derived from breast cancer, colorectal cancer, prostate cancer, ovarian cancer, leukemia, lymphomas, neuroblastoma, glioblastoma, melanoma, liver cancer, and lung cancer.

In a first step, the method of the present invention comprises the formation of a polymeric complex between one or more nucleic acid molecules to be delivered and one or more cationic carrier molecules, the carrier molecules facilitating intracellular uptake of the one or more nucleic acid molecules in the one or more target cells, wherein the complex becomes shielded by covalently attaching hydroxyalkyl starch to at least a part of the one or more carrier molecules.

The term “cationic carrier molecule”, as used herein, relates to any cationic (i.e. positively charged) compound having the capacity to bind or condense nucleic acid molecules, thus masking the negative charges of nucleic acids which aids to the passage through the plasma membrane of the target cells. In some embodiments, the carrier molecules are amphiphilic, that is, they possess both hydrophilic and lipophilic properties. Numerous cationic carrier molecules that may be employed in the present invention are well known in the art and commercially available, including inter alia cationic peptides protamines, (e.g., poly-arginines and poly-lysines), fusogenic peptides, polyamines, polyalkylenimines (e.g., polyethylenimine (PEI)), cationic dextrans, cationic cyclodextrins, chitosans, polyamidoamine dendrimers (PAMAM, Dendritech Inc.), cationic lipids (e.g., dioleoyltrimethyl-ammonium propane (DOTAP), N-[1(-2,3-dioleoyloxy)propyl]-N,N,N-trimethylammonium chloride (DOTMA), and N,N-dioleyl-N,N-dimethylammonium chloride (DODAC)), cationic cholesterol-complexes (e.g., PEI-cholesterol, poly-lysine-cholesterol), ready-to-use transfection reagents (e.g., Lipofectamine™, and Gene Portcr™), and combinations thereof. The term “one or more” carrier molecules, as used herein, does not only relate to the total number of carrier molecules present in a complex but also denotes that the polymeric complex may comprise only a single type of carrier molecules (e.g., polyethylenimine) or at least two different types of carrier molecules (e.g., polyethylenimine and poly-arginine).

In preferred embodiments, the one or more carrier molecules are selected from the group consisting of cationic lipids, cationic cholesterol-complexes, cationic peptides, in particular poly-arginines and poly-lysines, polyalkylenimines, in particular polyethylenimine, protamines, and combinations thereof. In a particularly preferred embodiment, the carrier molecule is polyethylenimine.

Typically, the polymeric complex formed between the nucleic acid molecules and the carrier molecules is accomplished via a non-covalent linkage.

The term “non-covalent linkage”, as used herein, refers to a variety of interactions that are not covalent in nature, between molecules or parts of molecules that provide force to hold the molecules or parts of molecules together usually in a specific orientation or conformation. Such non-covalent interactions include inter alia ionic bonds, hydrophobic interactions, hydrogen bonds, Van-der-Waals forces, and dipole-dipole bonds. In contrast, the term “covalent linkage”, as used herein, refers to an intra-molecular form of chemical bonding characterized by the sharing of one or more pairs of electrons between two components, producing a mutual attraction that holds the resultant molecule together.

The polymeric complexes used in the present invention are typically formed by mixing the one or more cationic carrier molecules and the one or more nucleic acid molecules in a suitable (physiological) buffer (e.g., PBS or HBG) at N/P ratios in the range between 1.0 (i.e. 1:1) and 20.0 (i.e. 20:1) but lower and higher ratios are possible as well. The N/P ratio is defined as the molar ratio of the nitrogen atoms present in the carrier molecules and the phosphate atoms present in the nucleic acid molecules. Preferably, the N/P ratio is in the range between 2.0 and 15.0 and particularly preferably in the range between 3.0 and 10.0, e.g., ratios of 3.2, 3.6, 4.0, 4.2, 4.6, 5.0, 5.2, 5.6, 6.0, 6.2, 6.6, 7.0, 7.2, 7.6, 8.0, 8.2, 8.6, 9.0, 9.2, and 9.6. The skilled person is well aware of the reaction conditions (e.g., temperature, pH) to be used for forming such polymeric complexes. Typically, such reactions are performed at room temperature and at a pH in the range between 6.5 and 7.5.

At least a part of one or more carrier molecules (that is, at least one of the carrier molecules) present in the polymeric complex are covalently attached to hydroxyalkyl starch. The term “starch”, as used herein, denotes a carbohydrate consisting of chains of glucose units coupled via α-1,4-glycosidic bonds and branched via α-1,6-glycosidic bonds.

In the context of the present invention, the term “hydroxyalkyl starch” (HAS) refers to a starch derivative which has been substituted by at least one hydroxyalkyl group, that is, a starch derivative in which at least one hydroxy group present anywhere, either in the terminal carbohydrate moiety and/or in the remaining part of the starch molecule. The alkyl group may be a linear or branched alkyl group. Typically, the hydroxyalkyl group contains 1 to 12 carbon atoms, preferably from 1 to 10 carbon atoms, more preferably from 1 to 6 carbon atoms, and most preferably 2-4 carbon atoms. In specific embodiments, the hydroxyalkyl starch used herein comprises two or more different hydroxyalkyl groups, for example hydroxyethyl (such as 1-hydroxyethyl, 2-hydroxyethyl) and hydroxypropyl (such as 1-hydroxypropyl, 2-hydroxypropyl, 3-hydroxypropyl) groups.

As long as the hydroxyalkyl starch remains soluble in water or aqueous buffers (e.g., physiological saline), the alkyl group may be mono- or poly-substituted with a halogen, an aryl group, and the like. Furthermore, the terminal hydroxy group of a hydroxyalkyl group, as defined herein, may be esterified or etherified. Numerous such derivatives are well known in the art (see, e.g., U.S. patent publication 2011/0054152 A1).

Examples of a “hydroxyalkyl starch” to be employed in the method of the present invention include inter alia hydroxymethyl starch, hydroxyethyl starch, starch hydroxypropyl, starch hydroxybutyl starch, hydroxypentyl starch, hydroxyhexyl starch, and so forth. Preferably, the term relates to hydroxyethyl starch, hydroxypropyl starch and hydroxybutyl starch, with hydroxyethyl starch being particularly preferred.

Hydroxyethyl starch (HES) can be characterized by its molecular weight distribution and its degree of substitution. There are two possibilities of describing the degree of substitution: (i) relatively to the portion of substituted glucose monomers with respect to all glucose moieties, and (ii) as the “molar substitution”, wherein the number of hydroxyethyl groups per glucose moiety are described. As used herein, the degree of substitution is given as “molar substitution”, as defined above.

Hydroxyethyl starch can be characterized by its molecular weight distribution and its degree of substitution solutions are usually present as polydisperse compositions, wherein each molecule differs from the other with respect to the polymerization degree, the number and pattern of branching sites, and the substitution pattern. Thus, HES is a mixture of compounds having different molecular weights. A particular HES solution is determined by its average molecular weight with the help of statistical means. In this context, the average molecular weight is calculated as the arithmetic mean depending on the number of molecules.

In the context of the present invention, the hydroxyethyl starch employed may have any average (mean) molecular weight of up to 1.000 kDa. Typically, the hydroxyethyl starch employed has an average molecular weight in the range between 1 kDa and 400 kDa, preferably in the range between 2 kDa and 300 kDa or between 3 kDa and 200 kDa, and particularly preferably in the range between 4 kDa and 100 kDa or between 5 kDa and 80 kDa. Examples of preferred ranges of average molecular weight are 4 kDa to 50 kDa, 10 kDa to 70 kDa, 12 kDa to 30 kDa, 10 kDa to 20 kDa, 20 kDa to 70 kDa, 40 kDa to 80 kDa, and 50 kDa to 100 kDa. Accordingly, the HES employed may have an average molecular weight inter alia of 4 kDa, 5 kDa, 8 kDa, 10 kDa, 12 kDa, 18 kDa, 20 kDa, 25 kDa, 30 kDa, 40 kDa, 50 kDa, 60 kDa, 70 kDa, 80 kDa, 90 kDa, and 100 kDa. It is also possible to use combinations of HES species having different average molecular weights, for example (10 kDa and 80 kDa) or (20 kDa and 70 kDa).

In the context of the present invention, the hydroxyethyl starch used may further exhibit an average (mean) molar degree of substitution (i.e. an average number of hydroxylethyl groups per glucose unit), which is typically in the range between 0.05 and 3.0, preferably in the range between 0.1 and 2.0 or between 0.1 and 1.5, and particularly preferably in the range between 0.1 and 1.0 or between 0.2 and 0.8. Examples of average molar degrees of substitution include inter alia 0.1, 0.2, 0.3, 0.4, 0.5, 0.6, 0.7, 0.8, 0.9, 1.0, 1.1, 1.2, 1.3, 1.4, and 1.5. It is also possible to use combinations of HES species having different average molar degrees of substitutions, for example (10 kDa and 80 kDa) or (20 kDa and 70 kDa). In the context of the present invention, the hydroxyethyl starch used may further exhibit an average degree of C2:C6 substitution with respect to the hydroxyethyl groups in the range between 2 and 20.

In preferred embodiments, the hydroxyethyl starch has:
(i) an average molecular weight in the range between 2 kDa and 300 kDa, and particularly in the range between 10 kDa and 200 kDa; and
(ii) an average number of hydroxylethyl groups per glucose unit in the range between 0.1 and 2.0, and particularly in the range between 0.1 and 1.0.

The hydroxyalkyl starch is covalently attached (i.e. bound) to the at least a part of the one or more cationic carrier molecules either directly or via a linker. Direct coupling can take place via the formation of a Schiff's base and subsequent reductive amination resulting in the formation of a methylene amine group. This synthesis scheme is well known in the art (see, e.g., Wollrab, A. (ed.) (2009) Organische Chemie, in: Springer Verlag Berlin, Heidelberg, p. 485-496). Alternatively, the hydroxyalkyl starch may be oxidized before coupling to a carrier molecule, where a specific oxidation of the reducing end groups is preferred to produce a reactive lactone (Hashimoto, H. et al. (1992) Kunststoffe, Kautschuk, Fasern, 9, 1271-1279.). Binding may also be accomplished by use of a linker. Any crosslinking agent may be used as a linker. Numerous crosslinking agents such as SMCC (succinimidyl-4-(N-maleimido-methyl) cyclohexane-1-carboxylate) are well known in the art (see, for example, international patent publication WO 2005/092928 A1 and U.S. patent publication 2011/0054152 A1) and commercially available.

In further specific embodiments, in the shielded polymeric complex the molar ratio between free and hydroxyalkyl starch (and preferably, hydroxyethyl starch)-modified carrier molecules is in the range between 1:99 and 99:1, and particularly in the range between 5:95 and 95:5. Exemplary ratios include inter alia 5:95, 8:92, 10:90, 12:88, 15:85, 18:82, 20:80, 30:70, 40:60, 50:50, 60:40, 70:30, 80:20, 82:18, 85:15, 88:12, 90:10, 92:8, and 95:5. In some typical embodiments, the ratios include inter alia 80:20, 82:18, 85:15, 88:12, 90:10, 92:8, and 95:5. A polymeric complex in which at least a part of the one or more carrier molecules are covalently attached to hydroxyalkyl starch is herein referred to as “shielded polymeric complex.”

The shielded polymeric complex is then allowed to get into contact with the one or more target cells. When performing an in vitro method, this may typically be accomplished by mixing the polymeric complex with or adding the polymeric complex to the target cells and subsequently incubating these mixtures in a suitable medium or buffer and under appropriate reaction. Depending on the type of target cells employed the skilled person is well aware how to select suitable conditions for performing such reactions. The term “to get into contact” with the target cells, as used herein, is to be understood as bringing the polymeric complex in close (spatial) proximity to the target cells, thus allowing the polymeric complex to associate with the target cells (but not necessarily to internalize into the target cells). It is also possible to directly administer the polymeric complex to a desired target site, for example a specific organ (e.g. liver, lung, eye, stomach, colon, and the like) or a tumor, and particularly a solid tumor or a cystic (i.e. fluid-filled) tumor. Such administration may be achieved by injecting a solution of the polymeric complex to the site of interest, for example, by using injection needles or by employing endoscopic surgical methods well known in the art. In case of administering the polymeric complex to a tumor, the polymeric complex may then passively diffuse through the cancerous tissue due to the leaky vasculature of tumors or inflammatory foci by enhanced permeability and retention (Maeda, H. et al. (2001) J. Contr. Release 74, 47-61; Iyer, A. K. et al. (2006) Drug. Discov. Today 11, 812-818). In addition, it is possible to administer the polymeric complex to the bloodstream of an organism and to transport it via the organism's circulatory system to the desired target site.

In order to facilitate the transport (i.e. delivery) of the shielded polymeric complex to the one or more target cells the shielded polymeric complex employed in the method may further comprise one or more targeting molecules. The term “targeting molecule”, as used herein, denotes any compound having the capability to specifically direct the polymeric complex to which it is attached to a particular target cell, e.g. by exhibiting binding activity for a surface molecule of the target cell. The one or more targeting molecules may be attached to the one or more nucleic acid molecules to be delivered and/or to the one or more carrier molecules, with an attachment to the latter being preferred. The attachment may be accomplished via a covalent linkage or a non-covalent linkage. The term “one or more” targeting molecules, as used herein, does not only relate to the total number of targeting molecules present in a complex but also denotes that the polymeric complex may comprise only a single type of targeting molecules (e.g., cell surface receptor-specific antibody fragment) or at least two different types of carrier molecules (e.g., an antibody fragment and a poly-arginine peptide).

Targeting moieties that are suitable for use in the present invention include sugars (e.g., fucose, galactose, and mannose), steroids, folic acid, viral peptides (e.g., HIV-TAT and HIV-REV, influenza HA2, polymyxin B), cell-penetrating peptides (e.g., poly-arginine and penetratin), transferrin, antibodies and fragments thereof (such as Fab fragments and single-chain antibodies), antibody-like compounds (such as anticalins) and specific cell-targeting peptides (e.g. RGD; reviewed in: Lochmann, D. et. al. (2004) J. Pharm. Biopharm. 58, 237-251). All these targeting molecules are well established in the art. The skilled person is also aware how to select and to attach one or more suitable targeting molecules in order to direct a given polymeric complex to a particular target cell (see also, e.g., Sambrook, J., and Russel, D. W. (2001), supra; Ausubel, F. M. et al. (2001), supra).

Subsequently, the polymeric complex is deshielded by removing the hydroxyalkyl starch attached to at least a part of the one or more cationic carrier molecules. Importantly, the hydroxyl starch (or at least a substantial portion thereof) present in the polymeric complex is only removed after the polymeric complex got in contact with (i.e. in close (spatial) proximity to) the one or more target cell. The term “substantial portion”, as used herein, refers to at least 30% or at least 40% of the hydroxyalkyl starch initially present in the polymeric complex, preferable to at least 50% or at least 60%, more preferably to at least 70% or at least 80%, and particularly at least 90% of the hydroxyalkyl starch initially present in the polymeric complex. Accordingly, the polymeric complex from which the hydroxyl starch has been removed is referred to herein as “deshielded complex”. The modification with hydroxyalkyl starch (preferably, hydroxyethyl starch) prevents the polymeric complex during transport to and delivery at the one or more target cells from aggregation, likely by masking the surface charges and decreasing non-specific electrostatic interactions, and also exhibits protection against degradation.

The hydroxyalkyl starch can be removed by any suitable means that allows specific removal of this modification, while leaving the remaining polymeric complex unchanged. For example, the hydroxyalkyl starch may be removed by chemical agents specifically cleaving the covalent bond (or any bond within a linker molecule employed) between the hydroxyalkyl starch and the one or more carrier molecules.

In preferred embodiments, the hydroxyalkyl starch is removed enzymatically, and in particular by exposing the shielded polymeric complex to amylase. The term “amylase”, as used herein, refers to enzymes that catalyze the breakdown of starch into sugars. More particularly, amylases are glycoside-hydrolases that act on α-1,4-glycosidic bonds. Amylases are classified in α-, β-, and γ-amylases: α-amylase (Enzyme Commission No. EC 3.2.1.1) is a 1,4-α-D-glucan glucanohydrolase which catalyses the random endohydrolysis of α-1,4-glycosidic linkages in amylase and amylopectin chains of starch, wherein reducing groups are released in α-configuration; β-amylase (Enzyme Commission No. EC 3.2.1.2) is a 4-α-D-glucan-maltohydrolase which catalyzes the hydrolysis of α-1,4-glycosidic linkages in amylase and amylopectin chains of starch so as to remove successively maltose units from the non-reducing ends of the chains; and γ-amylase (Enzyme Commission No. EC 3.2.1.3) is a glucan-1,4-α-glucosidase which catalyses the successive hydrolysis of terminal α-1,4-linked alpha-D-glucose residues from non-reducing ends of the amylase and amylopectin chains of starch with release of beta-D-glucose.

Within the present invention, any sub-type of amylase or any combinations thereof may be employed for deshielding the polymeric complex, that is, α-amylase, β-amylase, γ-amylase, α- and β-amylase, α- and γ-amylase; β- and γ-amylase, and α-, β-, and γ-amylase. In preferred embodiments, preparations of α-amylase only are used, which may be obtained from various organs or body fluids (e.g. pancreas and saliva) and from different organisms. Typically, the enzyme is purified from porcine resources and commercially available. Alternatively, the enzyme may be produced by means of recombinant DNA technology following protocols well established in the art (see, e.g., Sambrook, J., and Russel, D. W. (2001), supra; Ausubel, F. M. et al. (2001), supra).

However, instead of (or in addition to) amylase(s) other enzymes being capable of cleaving α-1,4-glycosidic bonds may be employed as well. An example of such enzyme is alglucosidase-alpha (Enzyme Commission No. EC 3.2.1.10) which has also α-1,6-glycosidic activity. Alglucosidase-alpha is commercially available.

In some embodiments, the amylase (or another enzyme(s) having α-1,4-glycosidic activity) is exogenously added to the one or more target cells. When performing an in vitro method, this may typically accomplished by mixing the amylase with or adding the amylase to the target cells, which are in contact with the shielded polymeric complex. It is also possible to directly administer the amylase to a desired target site, for example a specific organ (e.g. liver, lung, eye, stomach, colon, and the like) or a tumor, and particularly a solid tumor or a cystic (i.e. fluid-filled) tumor. Such administration may be achieved by injecting a solution of the amylase to the site of interest, for example, by using injection needles or by employing endoscopic surgical methods well known in the art. In addition, it is possible to administer the amylase to the bloodstream of an organism and to transport it via the organism's circulatory system to the desired target site. Optionally, the amylase may be attached to one or more targeting molecules as described above in order to facilitate specific delivery to a particular target cell. In one embodiment, the same one or more targeting molecules are employed for transport and delivery of both the polymeric complex and the amylase to a desired target site.

In some other embodiments, exposure to amylase is accomplished by the one or more target cells per se. In such case, the target cells are amylase-producing cells, in particular amylase-producing tumor cells. It is known that some types of tumor cells (such as lung cancer cells or ovarian cancer cells) produce considerable amounts of amylases, predominantly α-amylase. In specific embodiments, the use of amylase-producing tumor cells as target cells may be combined with a further exogenous addition of amylase.

The amylase is employed in a final concentration that is sufficient to remove at the target site at least a substantial portion (cf. the definition above) of the hydroxyalkyl starch attached to at least a part of the one or more cationic carrier molecules. In other words, within the present invention, a sufficient concentration of amylase has to be ensured at the target site, that is, in spatial proximity to the one or more target cells. Typically, the amylase is added in a final concentration of at least 20 U/I (e.g., 20, 30 or 40 U/I), at least 50 U/I (e.g., 50, 60 or 70 U/I) or at least 80 U/I (e.g., 80 or 90 U/I), and preferably of at least 100 U/I (e.g., 100 or 110 U/I). More preferably, the amylase is added in a final concentration of at least 120 U/I, such as a final concentration of 120 U/I, 125 U/I, 130 U/I, 135 U/I, 140 U/I, 145 U/I, 150 U/I, 155 U/I, 160 U/I, 165 U/I, 170 U/I, 175 U/I, 180 U/I, 185 U/I, 190 U/I, 195 U/I or 200 U/I. However, even higher final concentrations are possible, such as 210 U/I, 220 U/I, 230 U/I, 240 U/I, 250 U/I, 260 U/I, 270 U/I, 280 U/I, 290 U/I, 300 U/I, and so forth. In other embodiments, a final concentration of 400 U/I, 500 U/I, 600 U/I, 700 U/I or 800 U/I is used. Numerous methods are available in the art for determinating the amylase activity of a given sample (see, e.g., Vilijoen, A. and Twomey, P. J. (2007) J. Clin. Pathol. 60, 584-585; Bretaudiere, R. et al. (1981) Clin. Chem. 27, 806-815).

The amount of amylase required depends on various factors such as the amount (i.e. concentration) and composition of the polymeric complex (i.e. the amount of hydroxyalkyl starch present), the mode of administration of polymeric complex and amylase, the location of the target site relative to the site of administration, and the like. In specific embodiments, the extent of modification with hydroxyalkyl starch is indicative of the amount of amylase required for substantially removing the hydroxyalkyl starch. The higher the degree of molar substitution of the hydroxyalkyl starch, the more amylase is required to at least substantially remove the hydroxyalkyl starch. In some embodiments, an increase of the average molar degree of substitution by 0.1 (for example, from 0.7 to 0.8) results in an increase of the final concentration of amylase required to at least substantially remove the hydroxyalkyl starch (that is, to obtain the same results as with the lower degree of substitution) by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

Finally, the deshielded polymeric complex is allowed to internalize into the one or more target cells. This can be accomplished via passive cellular uptake or by applying any transfection protocol known in the art that is suitable for the target cells employed (see, e.g., Sambrook, J., and Russel, D. W. (2001), supra).

In some embodiments, the method of the present invention further comprises detecting the intracellular delivery (i.e. internalization) of the one or more nucleic acid molecules.

To this end, the one or more nucleic acid molecules (or at least a portion thereof) may be fused to one or more detectable labels. Labels that may be used herein include any compound, which directly or indirectly generates a detectable compound or signal in a chemical, physical or enzymatic reaction. Labeling and subsequent detection can be achieved by methods well known in the art (see, for example, Sambrook, J., and Russel, D. W. (2001), supra; and Lottspeich, F., and Zorbas H. (1998) Bioanalytik, Spektrum Akademischer Verlag, Heidelberg/Berlin, Germany). The labels can be selected inter alia from fluorescent labels, enzyme labels, chromogenic labels, luminescent labels, radioactive labels, haptens, biotin, metal complexes, metals, and colloidal gold. All these types of labels are well established in the art and can be commercially obtained from various suppliers. An example of a physical reaction that is mediated by such labels is the emission of fluorescence or phosphorescence upon irradiation. Alkaline phosphatase, peroxidase, β-galactosidase, and β-lactamase are examples of enzyme labels, which catalyze the formation of chromogenic reaction products, and which may be used in the invention.

In a further aspect, the present invention relates to the use of a method as defined herein for the transport (and controlled intracellular delivery) of one or more therapeutically active nucleic acid molecules into one or more target cells. In particular, this use of the method relates to the application of the one or more therapeutically active nucleic acid molecules that are delivered to the one or more target cells for the prevention and/or treatment of a condition selected from the group consisting of cancer, immune diseases, cardiovascular diseases, neuronal diseases, infections, and inflammatory diseases.

Accordingly, in another aspect the present invention relates to a pharmaceutical composition, comprising a shielded polymeric complex as defined herein above, and optionally further comprising amylase, for use in the prevention and/or treatment of a condition selected from the group consisting of cancer, immune diseases, cardiovascular diseases, neuronal diseases, infections, and inflammatory diseases.

The term “pharmaceutical composition”, as used herein, relates to a composition for administration to a subject, preferably to a human patient. Pharmaceutical compositions according to the present invention include any pharmaceutical dosage forms established in the art, such as inter alia capsules, microcapsules, cachets, pills, tablets, powders, pellets, lyophilisates, multi-particulate formulations (e.g., beads, granules or crystals), aerosols, sprays, foams, solutions, dispersions, tinctures, syrups, elixirs, suspensions, water-in-oil emulsions such as ointments, and oil-in water emulsions such as creams, lotions, and balms. The formulations may be packaged in discrete dosage units or in multi-dose containers.

In some embodiments, the pharmaceutical composition may represent a combination product or kit-of-parts comprising the polymeric complex (or a plurality of polymeric complexes) and the amylase packaged in different containers, wherein the dosage of the amylase is adapted to the physic-chemical and/or pharmacological properties of the polymeric complex (i.e. the molecular weight and the degree of hydroxylation of the hydroxyalkyl starch, and the like).

The pharmaceutical compositions of the invention include formulations suitable for oral, rectal, nasal, topical (including buccal and sub-lingual), peritoneal and parenteral (including intramuscular, subcutaneous and intravenous) administration, or for administration by inhalation or insufflation. Administration may be local or systemic.

The pharmaceutical compositions can be prepared according to established methods (see, for example, Gennaro, A. L. and Gennaro, A. R. (2000) Remington: The Science and Practice of Pharmacy, 20th Ed., Lippincott Williams & Wilkins, Philadelphia, Pa.; Crowder, T. M. et al. (2003) A Guide to Pharmaceutical Particulate Science. Interpharm/CRC, Boca Raton, Fla.; Niazi, S. K. (2004) Handbook of Pharmaceutical Manufacturing Formulations, CRC Press, Boca Raton, Fla.).

For the preparation of said compositions, one or more pharmaceutically acceptable (i.e.) inert inorganic or organic excipients (i.e. carriers) can be used. To prepare, e.g., pills, tablets, capsules or granules, for example, lactose, talc, stearic acid and its salts, fats, waxes, solid or liquid polyols, natural and hardened oils may be used. Suitable excipients for the production of solutions, suspensions, emulsions, aerosol mixtures or powders for reconstitution into solutions or aerosol mixtures prior to use include inter alia water, alcohols, glycerol, polyols, and suitable mixtures thereof as well as vegetable oils. The pharmaceutical composition may also contain additives, such as, for example, fillers, binders, wetting agents, glidants, stabilizers, preservatives, emulsifiers, and furthermore solvents or solubilizers or agents for achieving a depot effect. The latter is to be understood that the active peptides or compositions of the invention may be incorporated into slow or sustained release or targeted delivery systems, such as liposomes, nanoparticles, and microcapsules.

The pharmaceutical composition of the invention will be administered to the subject at a suitable dose. The particular dosage regimen applied will be determined by the attending physician as well as clinical factors. As is well known in the medical arts, an appropriate dosages for a given patient depend upon many factors, including the patient's size, sex, and age, the particular compound to be administered, time and route of administration, general health, pre-existing conditions, and other drugs being administered concurrently. The therapeutically effective amount for a given situation will readily be determined by routine experimentation and is within the skills and judgment of the ordinary clinician or physician. Generally, the dosage as a regular administration should be in the range of 1 μg to 1 g per day. However, a preferred dosage might be in the range of 0.01 mg to 100 mg, a more preferred dosage in the range of 0.01 mg to 50 mg and a most preferred dosage in the range of 0.01 mg to 10 mg per day.

The term “cancer”, as used herein, denotes any type or form of malignant neoplasm characterized by uncontrolled division of target cells based on genetic re-programming and by the ability of the target cells to spread, either by direct growth into adjacent tissue through invasion, or by implantation into distant sites by metastasis (where cancer cells are transported through the bloodstream or lymphatic system). Examples include inter alia breast cancer, colorectal cancer, prostate cancer, ovarian cancer, leukemia, lymphomas, neuroblastoma, glioblastoma, melanoma, liver cancer, and lung cancer.

The term “immune disease”, as used herein, refers to any disorder of the immune system. Examples of such immune diseases include inter alia immunodeficiencies (i.e. congenital or acquired conditions in which the immune system's ability to fight infectious diseases is compromised or entirely absent, such as AIDS or SCID), hypersensitivity (such as allergies or asthma), and autoimmune diseases. The term “autoimmune disease”, as used herein, is to be understood to denote any disorder arising from an overactive immune response of the body against endogenic substances and tissues, wherein the body attacks its own cells. Examples of autoimmune diseases include inter alia multiple sclerosis, Crohn's disease, lupus erythematosus, myasthenia gravis, rheumatoid arthritis, and polyarthritis.

The term “cardiovascular disease”, as used herein, refers to any disorder of the heart and the coronary blood vessels. Examples of cardiovascular diseases include inter alia coronary heart disease, angina pectoris, arteriosclerosis, cardiomyopathies, myocardial infarction, ischemia, and myocarditis.

The term “neuronal disease” (or “neurological disorder), as used herein, refers to any disorder of the nervous system including diseases of the central nervous system (CNS) (i.e. brain and spinal cord) and diseases of the peripheral nervous system. Examples of CNS diseases include inter alia Alzheimer's disease, Parkinson's disease, Huntington's disease, Locked-in syndrome, and Tourettes syndrome. Examples of diseases of the peripheral nervous system include, e.g., mononeuritis multiplex and polyneuropathy.

The term “infection”, as used herein, refers to any disorder based on the colonization of a host organism by a foreign pathogen such as bacteria, viruses or fungi. Examples of bacterial infections include inter alia bacterial meningitis, cholera, diphtheria, listeriosis, whooping cough, salmonellosis, tetanus, and typhus. Examples of viral infections include inter alia common cold, influenza, dengue fever, Ebola hemorrhagic fever, hepatitis, mumps, poliomyelitis, rabies, and smallpox. Examples of fungal infections include inter alia tinea pedis, blastomycosis, and candidiasis.

The term “inflammatory disease”, as used herein, refers to any disorder associated with inflammation including, e.g., acne, asthma, hay fever, arthritis, inflammatory bowel disease, pelvic inflammatory disease, and transplant rejection.

In a further aspect, the present invention relates to a method for the prevention and/or treatment of a condition selected from the group consisting of cancer, immune diseases, cardiovascular diseases, neuronal diseases, infections, and inflammatory diseases, comprising: administering a pharmaceutical composition of the invention to a subject. Preferably, the subject is a human patient.

The invention is further described by the figures and the following examples, which are solely for the purpose of illustrating specific embodiments of this invention, and are not to be construed as limiting the scope of the invention in any way.

### EXAMPLES

### Example 1

### Materials and Methods

### 1.1 Materials

Hydroxyethyl starch having an average molar mass of 70 kDa (HES70) and a molar substitution of 0.5 (i.e. the mean number of hydroxyethyl groups per glucose unit) was provided by Serumwerk Bernburg, Germany. Hydroxyethyl starch having an average molar mass of 20 kDa (HES20) was generated by acid hydrolysis of HES70 (cf. below). Linear polyethylenimine with an average molar mass of 22 kDa (PEI22) and the PEI22-PEG20 conjugate (PEG20: polyethylene glycol having an average molecular weight of 20 kDa) were synthesized in the lab of Prof. Dr. Ernst Wagner (Department of Pharmacy, LMU Munich). α-amylase (AA) from porcine pancreas (30 U/mg amylase), Triton-X 100, and citrated human plasma were purchased from Sigma-Aldrich (Steinheim, Germany). Sodium cyano-borohydride (NaBH₃CN) was obtained from Merck Schuchardt (Hohenbrunn, Germany). Plasmid pCMVluc was purchased from PlasmidFactory, Bielefeld, Germany. Phadebas® Amylase Test was purchased from Magle AB Lund, Sweden. Label IT® Cy5 Labeling Kit was obtained from Mirus Bio Corporation. Other solvents and chemicals were reagent grade and were used as received. Blood from C57BL/6 mice was obtained from the Institute of Pharmacology, Department of Pharmacy, LMU Munich.

For cell culture experiments, murine neuroblastoma cells (N2A) and human hepatoma cells (HUH7) were obtained from the Deutsche Sammlung von Mikroorganismen and Zellkulturen (Braunschweig, Germany) and cultured at 37° C. and 5% CO2 atmosphere in Dulbecco's Modified Eagle Medium (DMEM) and in DMEM/HAM's F12 medium (1:1), respectively. All media were supplemented with 10% (v/v) FCS, 4 mM glutamine, 100 U/ml penicillin, and 100 μg/ml streptomycin (all purchased from Life Technologies, Karlsruhe, Germany).

### 1.2 Acid Hydrolysis of HES70

HES70 was hydrolyzed according to the protocol disclosed in U.S. Pat. No. 5,424,302 (Laevosan GmbH, Austria) with some modifications. Briefly, 5 g HES70 were dissolved in 100 ml 0.05M HCl and heated to 100° C. in an oil bath (reflux condensation). The reaction was stopped after 2 h by addition of 1 M sodium hydroxide (NaOH) solution, and adjusting the pH to neutrality. The resulting solution was dialyzed for 48 hours against highly purified water (CelluSep T1, nominal MWCO 3500 Da; Membrane Filtration Products Inc, Seguin, Tex., USA). Then, the product was lyophilized and stored in a desiccator at room temperature.

### 1.3 Determination of the Molar Mass of Acid-Hydrolyzed HES Using AF4

The asymmetric flow field flow fractionation (AF4) instrument used consisted of an Eclipse 2 separation system that was coupled to an 18 angle multi-angle light scattering (MALS) detector (DAWN EOS MALS; both from Wyatt Technology Corp., Santa Barbara, Calif.; USA) with a laser of wavelength 690 nm. In addition, the Agilent HPLC system 1100 Series was used (Agilent Technologies, Palo Alto, Calif., USA). It was equipped with a degasser, isopump, autosampler, and the refractive index (RI) detector.

Samples were prepared at a concentration of 5 mg/m in 50 mM NaCl solution (filtered with a 0.1 μm cellulose nitrate filter, Whatman GmbH Dassel, Germany) supplemented with 0.02% w/v NaN₃ as preservative. 100 μl HES samples were injected into the standard separation channel system (25 cm), with channel thickness of 350 μm and equipped with a 5 kDa cutoff regenerated cellulose ultrafiltration membrane (Wyatt Technology Europe, Dernbach, Germany). Separation was performed with an applied channel flow of 2 ml/min coupled to a linearly decreasing cross flow gradient (from 2 ml/min to 0 ml/min over 30 minutes). Molecular weight was calculated using ASTRA software version 5.3.2.22 (Wyatt Technology Corp.) using the refractive index increment (dn/dc) of 0.1475 cm³/g for HES.

### 1.4 Biodegradation of HES70 and HES20 with Pancreatic α-Amylase

The enzymatic activity of pancreatic α-amylase (AA) was determined using the Phadebas® Amylase test according to the manufacturer's instructions. Mixtures of HES70 or HES20 with pancreatic α-amylase were prepared at a HES concentration of 5 mg/ml in HEPES buffered glucose (HBG: 20 mM HEPES, 5% (w/v) glucose, pH 6.0 or pH 7.1) or in phosphate buffered saline (PBS: 136.89 mM NaCl, 2.68 mM KCl, 8.10 mM Na₂HPO₄, 1.47 mM KH₂PO₄, pH 7.4). 100 μl samples were injected into the AF4 channel assembly. All samples containing AA were adjusted to an enzyme activity of 40 U/I or 100 U/I (the latter value corresponds to the serum amylase activity of healthy adults (Junge, W. et al. (2001) Clin. Biochem. 34, 607-615).

The reaction mixtures were incubated at 25° C. or 37° C., and aliquots were collected after 0, 0.5, 1, 2, 4, 6, and 24 h, respectively. In order to stop the enzymatic degradation of HES, the aliquots were heated to 99° C. for 3 min. The assay was performed under aseptic conditions with sterile filtration to prevent possible degradation caused by microbial contamination. The reduction in the molar mass of HES70 and HES20 was monitored by using the Wyatt Eclipse 2 AF4 system in combination with MALS and RI-detection as described above.

### 1.5 Preparation of HES-PEI Conjugates

Conjugates of PEI22 with either one of HES70 and HES20 were prepared in a molar ratio HES:PEI of 25:1 as described (Kircheis R. et al. (2001) Gene Ther. 8, 28-40). PEI was coupled to HES via the formation of a Schiff's base and subsequent reductive amination. To this end, 50 mg linear PEI22 were added to HES20 or HES70 in 150 mM PBS buffer (pH 7.4, and agitated at room temperature. After 2 hours, 59.8 mg of the reducing agent NaBH₃CN were added, and reductive amination was performed for 20 h. Unbound HES was removed by ion exchange chromatography. Mixtures of HES and PEI were loaded onto a cation-exchange column (Bio-Rad Macro-Prep high S HR 10/10; Hercules, Calif., USA) and fractionated using a sodium chloride gradient from 0.5 M to 3.0 M in 20 mM HEPES, pH 7.3. PEI-containing fractions were detected by UV-spectroscopy at 280 nm. The collected fractions were dialyzed against highly purified water (CelluSep T1, nominal MWCO 3500 Da; Membrane Filtration Products Inc, Seguin, Tex., USA) and lyophilized.

### 1.6 Nuclear Magnetic Resonance Spectroscopy

The HES-PEI conjugates were characterized with respect to coupling efficiency and molar ratio by nuclear magnetic resonance spectroscopy. For the ¹H-NMR measurements, 10 mg of HES20-PEI22 and HES70-PEI22 were dissolved in D₂O (“heavy water” enriched in deuterium), and spectra were obtained by using a JNMR-GX500 (500 MHz) spectrometer (Jeol GmbH, Eching, Germany).

### 1.7 Copper Assay

The assay was aimed at determining the concentration of PEI in the respective HES-PEI conjugates and was performed as described (Ungaro, F. et al. (2003) J. Pharm. Biomed. Anal. 31, 143-149). In brief, a calibration curve for PEI was generated (concentration range 5.0-50.0 μg/ml, in 150 mM PBS, pH 7.4) and analyzed photometrically at 285 nm. 23 mg CuSO₄.×5 H₂O were dissolved in 100 ml 0.1 M NaAcetate buffer (pH 5.4), added to the HES-PEI conjugates (or free PEI as control), and incubated at room temperature for 15 min. Photometric analysis was performed on an Agilent 8453 UV-vis Spectroscopy System (Agilent Technologies, Waldbronn, Germany).

### 1.8 Size Exclusion Chromatography

Characterization of the particle size and surface charge of HES-PEI conjugates was performed using a combination of size exclusion chromatography (SEC) multi-angle light scattering (MALS). HES20-PEI22 and HES70-PEI22 were employed at a concentration of 5 mg/ml (in 50 mM NaCl). Control samples (mixture of HES and PEI as well as free PEI) were used at a PEI concentration of 1 mg/ml (in 50 mM NaCl). SEC was performed with 100 μl samples per SEC run by means of a TSKgel G5000PWXL-CP column (7.8 mm×30.0 cm; Tosoh Bioscience GmbH, Stuttgart, Germany) at a flow rate of 0.5 ml/min. MALS was performed at 18 angles using the Eclipse 2 separation system (Wyatt Technology Corp. Santa Barbara, Calif., USA) and the 1100 Series Agilent HPLC system (Agilent Technologies, Palo Alto, Calif., USA). For the generation of chromatograms data analysis was done by means of the ASTRA software package (version 5.3.2.22, Wyatt Technology Corp).

### 1.9 Quartz Crystal Microbalance with Dissipation (QCM-D)

A Q-Sense E4 instrument (Q-Sense, Gothenburg, Sweden) was used for the analysis of the enzymatic degradation of HES in different HES-PEI conjugates. Prior to each measurement, the silica-coated QCM-D sensor crystals (QSX 303, Q-Sense) were washed with 2% SDS solution and treated with oxygen plasma (0.4 mbar, 150 W) for 45 minutes (TePla 100 System, Feldkirchen, Germany) in order to decontaminate the crystal surface. The system was operated at 25° C. in the flow mode, interrupted by phases of no flow. A single QCM-D run included the following five sections: (1) rinsing of the system with buffer (15 min); (2) polymer adsorption onto the SiO₂ sensor (5 min sample flow, 10 min without flow); (3) rinsing of the system with buffer (15 min); (4) start of enzymatic degradation by adding α-amylase (5 min sample flow, 55 min without flow); and (5) rinsing of the system with buffer (15 min).

A series of different HES-PEI conjugates was prepared and tested with the special emphasis on the molar mass and the degree of hydroxyethylation of HES: HES30-PEI [0.4]/[1.0]; HES60-PEI [0.7]/[1.0]/[1.3]; and HES70-PEI [0.5], where the numbers after HES represent the average molar mass, and those in square brackets the degree of molar substitution. Naked PEI (LPEI) and PEG20-PEI served as controls. All polymers were applied at a concentration of 100 μg/ml (based on LPEI) in HBG pH 7.1. The enzyme activity was set to 100 U/I and 300 U/I, respectively (according to Phadebas® Amylase Test). BSA was used as negative control. The Sauerbrey model (Sauerbrey, G. (1959) Zeitschrift für Physik 155, 206-222) was used to monitor the adsorbed and desorbed mass onto the silica-coated quartz crystal. Changes of mass Δm [ng/cm²] on the quartz surface are defined as:

$$Δ m = - \frac{C \times Δ f}{n}$$

wherein;
C is the mass-sensitivity constant (17.7 ng Hz⁻¹ cm⁻² for the 5 MHz quartz crystal)
Δf [Hz] is the resonance frequency and
n=1, 3, 5, 7 is the overtone number.

In the present analysis, the low overtone number 3 was used to avoid underestimation of the mass. QSoft 4.01 software was used for data acquisition, QTools for data analysis (both from Q-Sense, Sweden).

### 1.10 Preparation of Polymeric Complexes

Polymeric complexes (herein also referred to as “polyplexes”, “naked Px” or “nPx”) were prepared via the rapid addition and mixing of PEI to pCMVLuc plasmid DNA (pDNA) (final DNA concentration of 20 μg/ml for in vitro experiments or 200 μg/ml for in vivo analyses, each in HBG, pH 7.1) at N/P ratios of 3.6, 4.8, 6.0, 7.2, and 8.0 (i.e. the molar ratio of PEI nitrogen atoms to pDNA phosphate atoms), and then incubated at room temperature (RT) for 30 minutes prior to analysis. For example, PEI/DNA complexes with an N/P ratio of 6.0 were composed of 20 μg pDNA and 16 μg PEI.

HESylated polymeric complexes were produced in an analogous manner but by partially replacing PEI with HES-modified PEI. For example, HES70-PEI/DNA complexes with an N/P ratio 6.0 and a ratio of PEI to HES-modified PEI of 90:10 were made of 20 μg DNA, and a mixture of 14.4 μg PEI and 1.6 μg HES70-PEI22 (weight of the PEI fraction). HES70-PEI/DNA (“HES70Px”) and HES20-PEI/DNA (“HES20Px”) complexes were each generated with PEI:HES-PEI ratios of 95:5, 90:10, and 85:15, respectively. PEG20-PEI complexes as controls (“PEG20Px”) were prepared with a PEI:PEG-PEI ratio of 90:10.

### 1.11 Determination of Particle Size and Zeta Potential

The analysis of the particle size and surface charge (via the determination of the zeta potential) of various polyplexes (nPx, HES70Px, HES20Px, and PEG20Px) was performed in HBG, pH 6.0 or pH 7.1 using a Malvern Zetasizer Nano ZS (Malvern Instruments, Worcestershire, United Kingdom). Experiments were carried out at 25° C. or 37° C. in semi-micro PMMA disposable cuvettes (Brand GmbH, Wertheim, Germany) and in folded capillary cells (Malvern Instruments, Worcestershire, United Kingdom). For data analysis, the viscosity of the dispersant (water with 5% (w/v) glucose) was set as 1.0366 mPas at 25° C., and 0.8359 mPas at 37° C. The data obtained with respect to the particle size distribution are means of at least three independent measurements (n≧3), wherein each measurement comprises three serial runs of 15 sub-runs. The subsequent analysis of the particles' surface charge was carried out in triplicate without further treatment of the samples. Voltage was set to 100 V, and a monomodal setup was applied. Malvern Zetasizer software version 6.12 (also from Malvern Instruments) was used for data acquisition and analysis.

### 1.12 Treatment of Polymeric Complexes with Pancreatic α-Amylase

Naked polyplexes (nPx) and HES70Px polyplexes were prepared using a DNA concentration of 20 μg/m in HBG, pH 6.0, and at an N/P ratio of 6.0. In case of HESylated polyplexes varying ratios of PEI:HES-PEI (95:5, 90:10, and 85:15) were employed. The polymeric complexes were incubated for 30 min at room temperature. Afterwards, an α-amylase (AA) stock solution was added to the respective polyplexes, resulting in a final AA concentration of 40 U/I or 100 U/I. The samples were mixed intensively and immediately analyzed using a Malvern Zetasizer Nano ZS. Analysis of particle size and zeta potential of PEI/DNA complexes was performed at various time points of 0, 0.25, 0.5, 1, 2, 4, and 6 h while the polyplexes were kept at 25° C. or 37° C.

### 1.13 Erythrocyte Aggregation Assay

Blood from 3 months old male C57BL/6 mice (obtained from the Department of Pharmacy, Institute of Pharmacology, LMU Munich) was collected, spiked with 3.2% (m/v) sodium citrate to prevent coagulation, and washed six times by centrifugation (2500×g, 10 min, 4° C.) with PBS, pH 7.4, until a colorless supernatant was obtained. The erythrocytes obtained were re-suspended in phosphate-buffered saline at a concentration of 2% (v/v). 50 μl HES70Px or HES20Px in HBG, pH 7.1 (final concentration of 1 μg pDNA, N/P ratio of 6.0, and PEI:HES-PEI ratios of 95:5, 90:10, and 85:15, respectively) were mixed with 100 μL erythrocyte suspension in PBS pH 7.4 in the presence or absence of α-amylase (“AA”). If applicable, AA was added in a final concentration of 40 U/I. Buffer, buffer+AA, naked PEI-DNA complexes (“nPx”) and PEG20Px (both as in FIG. 5) were used as controls. The solutions were incubated in 24-well plates (Corning Costar; Sigma-Aldrich, Steinheim, Germany) for 90 min at 37° C. under constant gentle agitation. For microscopic analysis, pictures were taken with a Keyence VHX-500F digital microscope (Keyence Corporation, Osaka, Japan) with a 1000-fold magnification.

### 1.14 Luciferase Reporter Gene Expression

In vitro pDNA transfection efficiency was evaluated in murine N2A neuroblastoma and human HUH7 hepatoma cell lines. Experiments were performed in 96 well plates by seeding 24 h prior to transfection about 1×10⁴ cells per well in 100 μl medium. Immediately before transfection, the medium was replaced with fresh medium supplemented with or lacking porcine pancreatic α-amylase (40 U/I or 100 U/I). 10 μl of the respective polyplex solutions (20 μg/ml DNA, N/P ratio of 6.0) were added to the cells. 4 h after transfection, the medium was replaced with fresh medium supplemented with or lacking α-amylase. 24 h after transfection, the cells were treated with 100 μl cell lysis buffer (25 mM Tris, pH 7.8, 2 mM EDTA, 2 mM DTT, 10% (v/v) glycerol, 1% (v/v) Triton X-100). Luciferase activity was determined in 35 μl cell lysate using a commericially available kit (Luciferase Assay System, Promega, Mannheim, Germany) on a luminometer for 10 s (Centro LB 960 instrument, Berthold, Bad Wildbad, Germany).

### 1.15 Metabolic Activity of Transfected Cells

Metabolic activity of the transfected N2A and HUH7 cells was analyzed by means of a MTT assay (Sigma-Aldrich, Steinheim, Germany). 24 h after transfection, per well of a microtiter plate 10 μl MTT (3-(4,5-dimethylthiazol-2-yl)-2,5-diphenyltetrazolium bromide; 5 mg/ml in PBS) were added to the cell suspension, thus resulting in a final concentration of 0.5 mg/ml MTT. After incubation for 2 h, excess dye was removed, and the cells were lyzed by incubation at −80° C. for 30 min. The formazan reaction product was dissolved in 100 μl dimethyl sulfoxide (DMSO) and quantified by a plate reader (Tecan, Groedig, Austria) at a wavelength of 590 nm with background correction at 630 nm. The metabolic activity (in % relative to control wells containing HBG treated cells) was calculated as “Aₜₑₛₜ/A_{control×100”.}

### 1.16 Flow Cytometry

The uptake of HES20Px and HES70Px (each with PEI:HES-PEI ratios of 90:10; prepared as described in FIG. 5) and of control particles was studied in N2A neuroblastoma cells in the presence or absence of 40 U/I α-amylase. Aliquots of 1×10⁵ cells seeded in 24-well plates 24 h prior to transfection. Directly before transfection, the medium was exchanged against fresh medium with/without pancreatic AA. 50 μl of the respective polyplexes (N/P ratio 6.0; μg/ml pDNA, 10% of which is Cy5-labeled) were added per well. For binding studies, the treated cells were kept at 4° C. for 30 min. Samples used for the determination of cellular uptake were incubated at 37° C. for 60 min. After washing the cells with PBS (phosphate buffered saline), the polyplexes were disassembled by adding 1000 I.E./ml heparin, and the cells were trypsinated. The percentage of Cy5 positive cells was determined by measuring the excitation of Cy5 at 635 nm. The mean fluorescence intensity (“MFI”) was determined by measuring the emission of Cy5 at 665 nm.

### 1.17 Systemic Treatment of Tumor-Bearing Mice with HESylated pDNA Polyplexes

In vivo pDNA expression studies were evaluated in N2A tumor-bearing A/J mice (6-8 weeks, female, Harlan Winkelmann). 1×10⁶ N2A cells in 100 μl PBS were inoculated subcutaneously into the flank of each mouse. Once the tumors reached the desired size of approximately 100 mm³, polymeric complexes were systemically administered via the tail vein. LPEI-based pDNA complexes were generated at an N/P ratio of 6.0 with a pCMVluc amount of 50 μg per mouse in an injection volume of 250 μl (HBG, pH 7.4).

In order to study the effect of different HES70 amounts on luciferase gene expression in the lung and in the tumor HESylated polyplexes (n=4) were prepared with 10%, 30% and 50% shielding agent HES70PEI, corresponding to 4 μg (10%), 12 μg (30%) and 20 μg (50%) HES70PEI based on the amount of LPEI, respectively. Naked LPEI and PEGylated (30% PEG20PEI, based on LPEI) particles served as controls. Furthermore, in order to investigate the impact of the degree of hydroxyethylation of HES on the lung/tumor expression non-biodegradable HES60[1.3]- and PEG20-containing particles (n=5) were prepared in the same molar ratio (HES-PEI to PEI).

In all experiments, animals were sacrificed 24 h after injection of the polymeric complexes, and lung and tumor tissues were resected and stored at −80° C. Tissues were homogenized in cell culture lysis reagent (25 mM Tris, pH 7.8, 2 mM EDTA, 2 mM DTT, 10% glycerol, 1% Triton X-100) using a tissue and cell homogenizer (MP, FastPrep®-24, Solon, Ohio, United States), followed by a centrifugation step at 3000 g and 4° C. for 10 min to separate insoluble cell components. 50 μl of the supernatants were transferred to white 96 well plates (TPP, Trasadingen, Switzerland) and luciferase activity was determined using a luciferase assay kit (100 μl Luciferase Assay Buffer, Promega, Mannheim, Germany) and a Centro LB 960 luminometer (Berthold, Bad Wildbad, Germany). Luciferase transfection performance is expressed as relative light units (RLU) per mg organ.

### Example 2

### Preparation and Biophysical Characterization of Polymeric Complexes

### 2.1 Acid-Induced Fragmentation of HES70

In order to test the stability of hydroxyethyl starch fractions having different molecular weights were subjected to acid-induced fragmentation. Treatment of HES70 for 2 h using 1 M HCl or 0.1 M HCl resulted in a very rapid degradation with concomitant difficulties in controlling the molar mass of the fragmentation products. In contrast, carrying out the hydrolysis reaction using 0.05 M HCl resulted in a fragmentation product of around 20 kDa with low polydispersity (i.e. molecular weight distribution of the fragmentation products) of 1.15, as determined by AF4-MALS. The overall yield was 62% (w/w). Accordingly, the molecular weight of HES appears to have significant impact on the “shielding capacity” of HES.

### 2.2 HES Degradation Experiments with Pancreatic α-Amylase

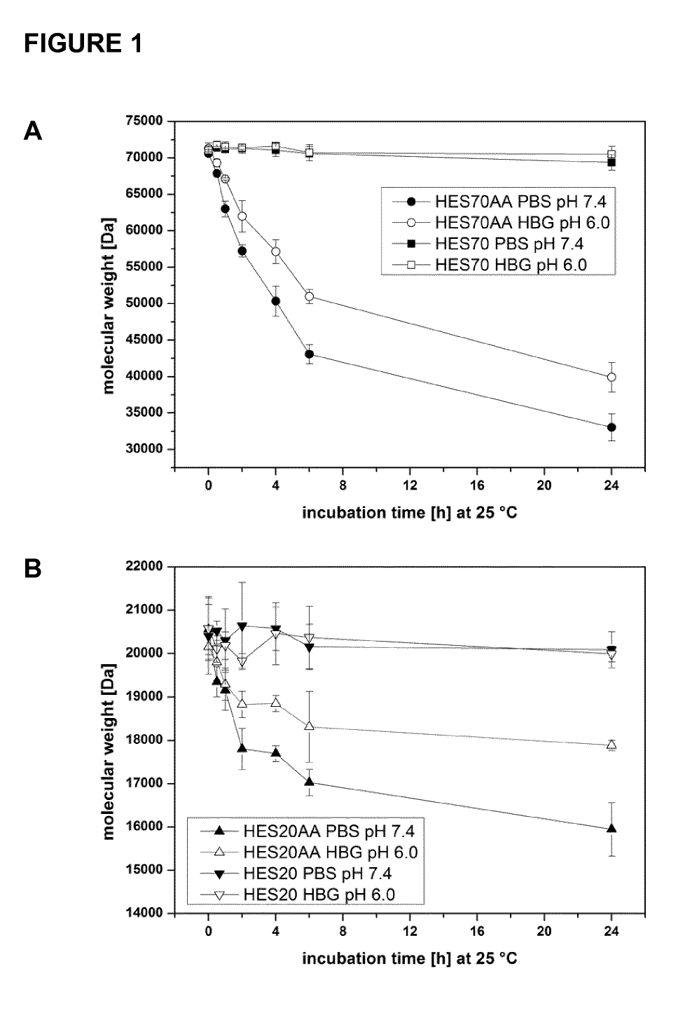

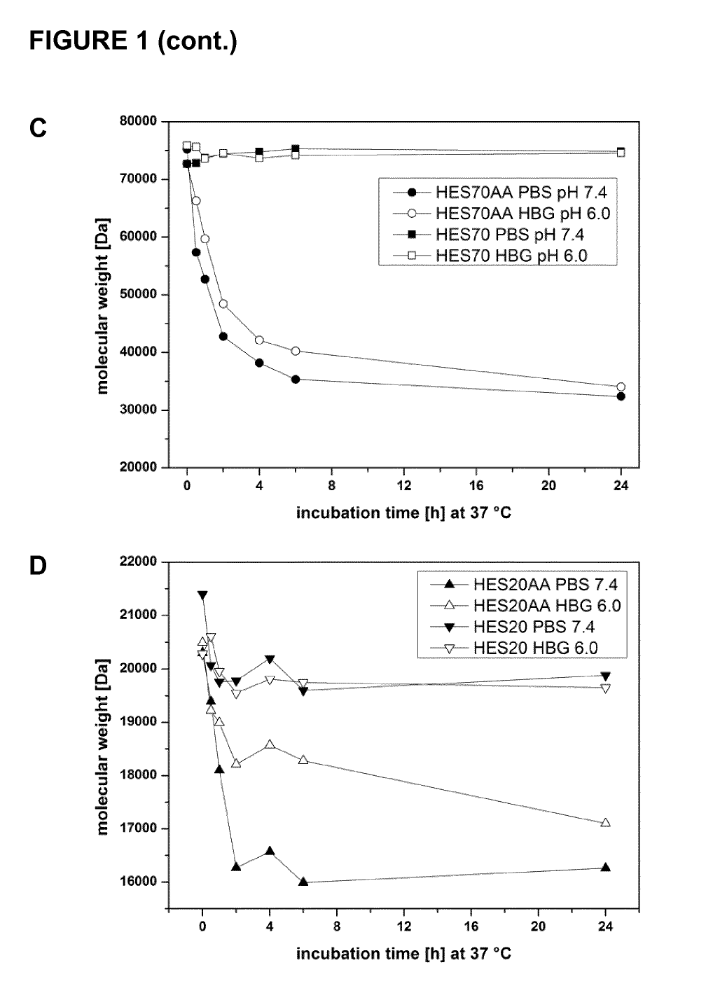

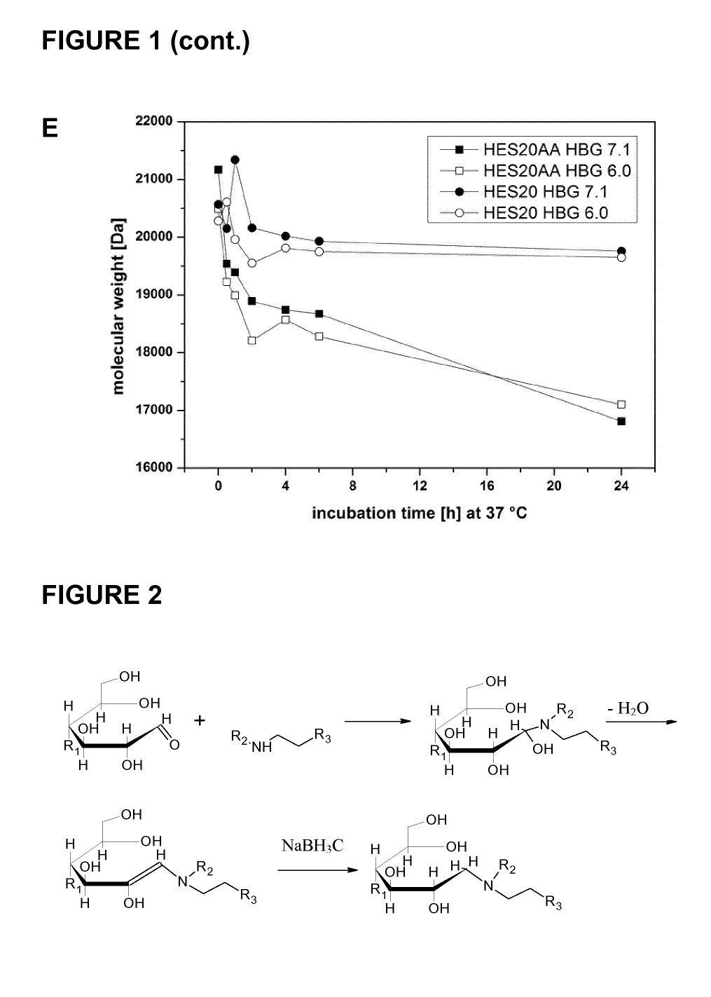

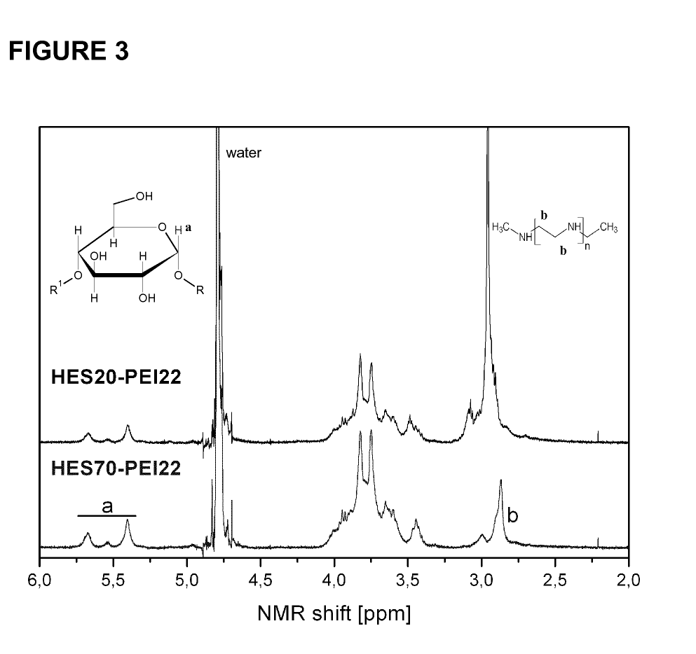

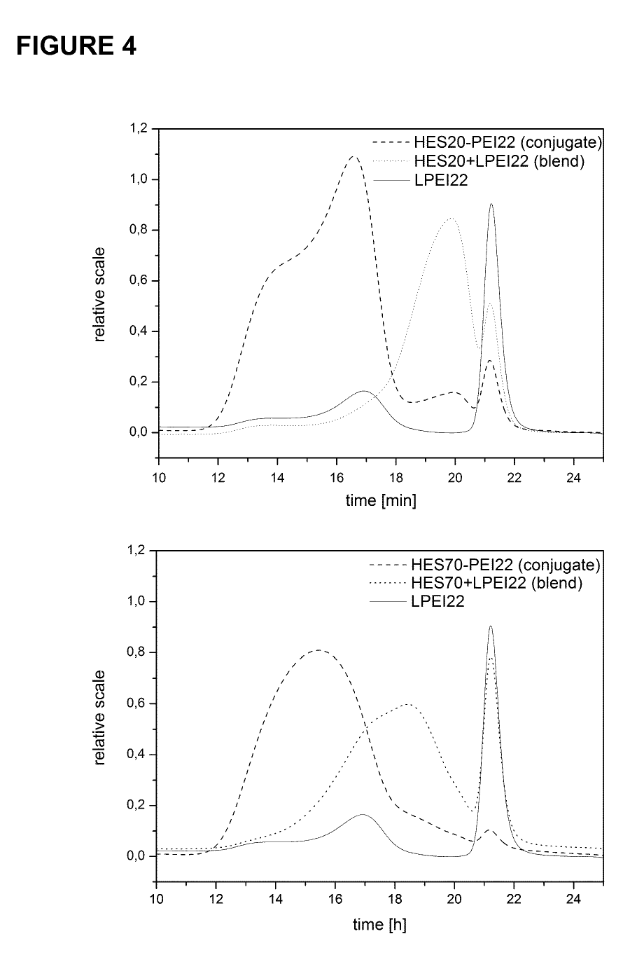

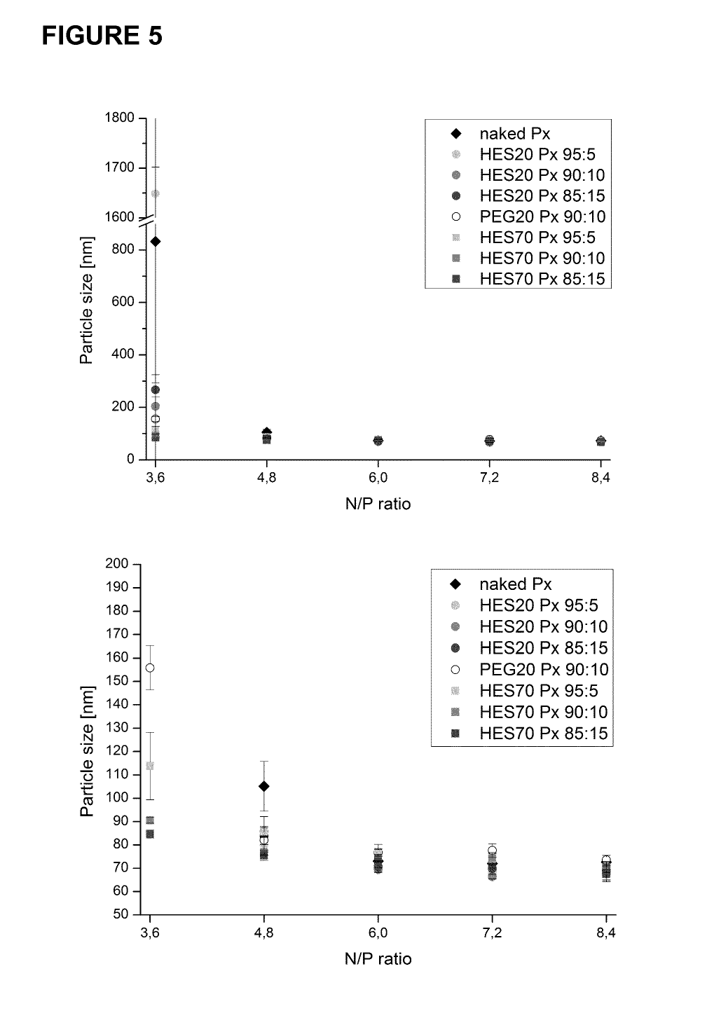

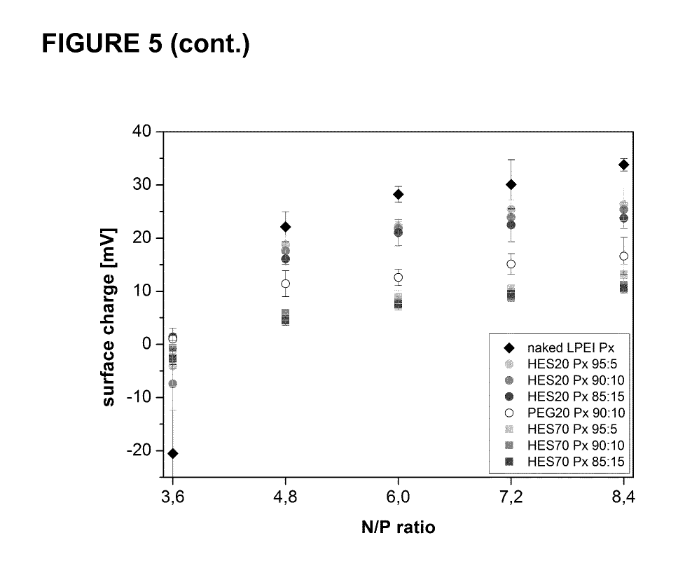

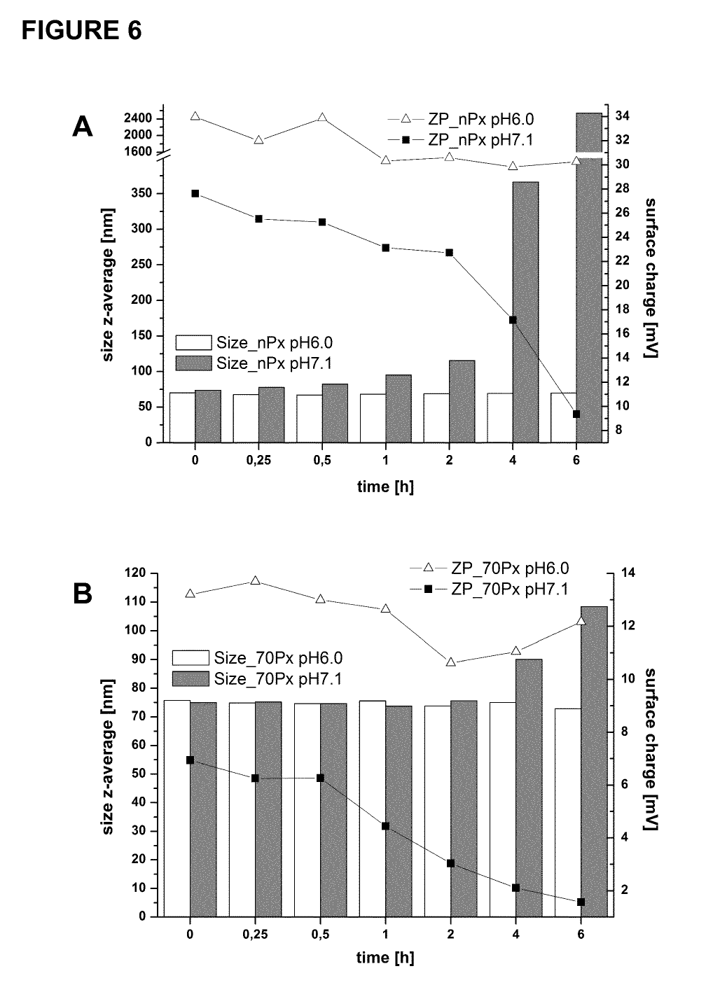

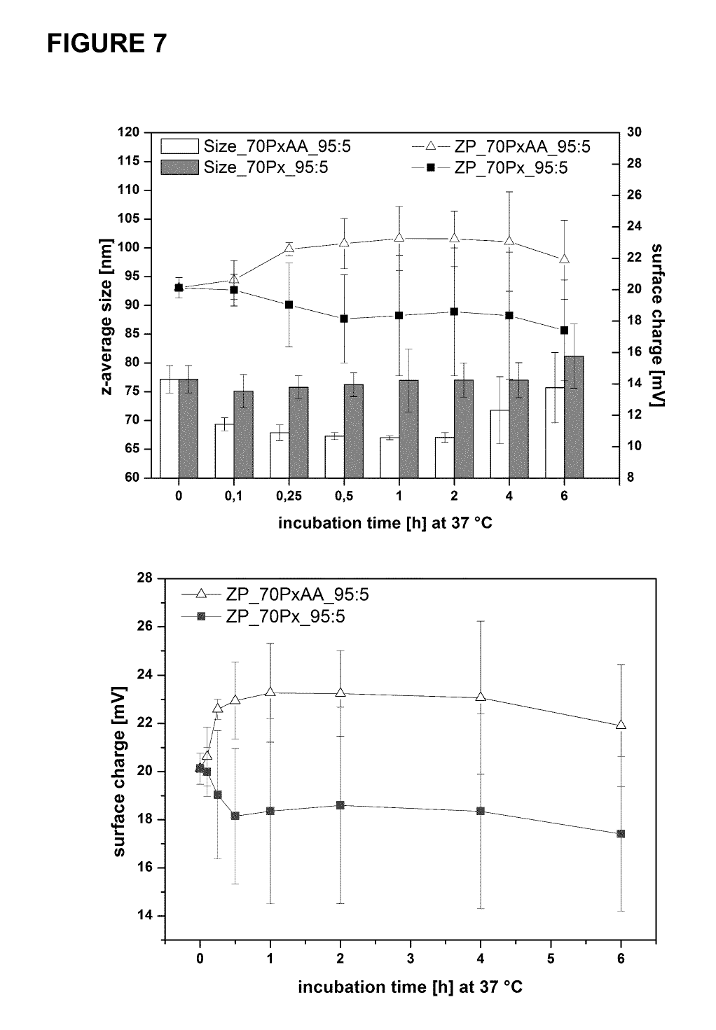

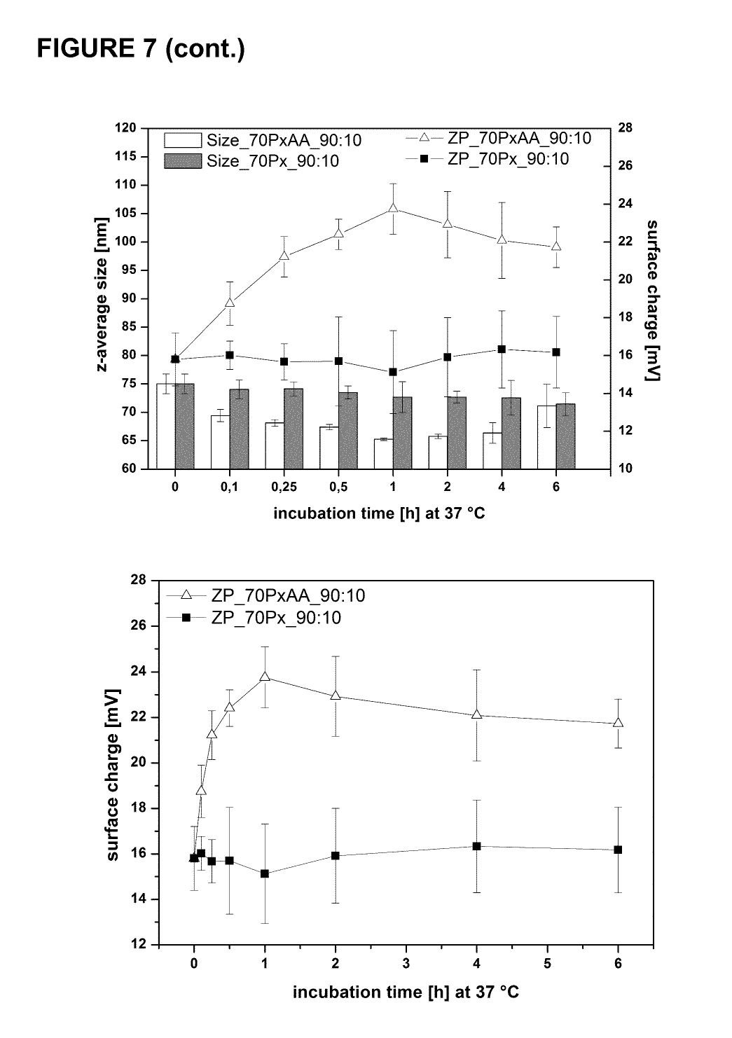

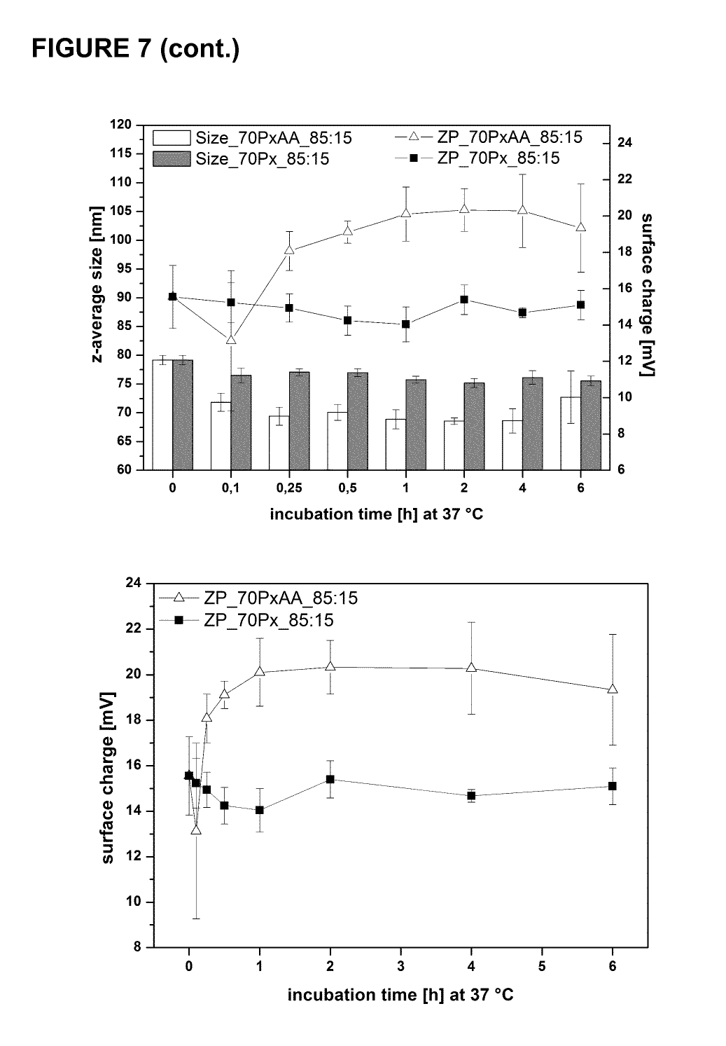

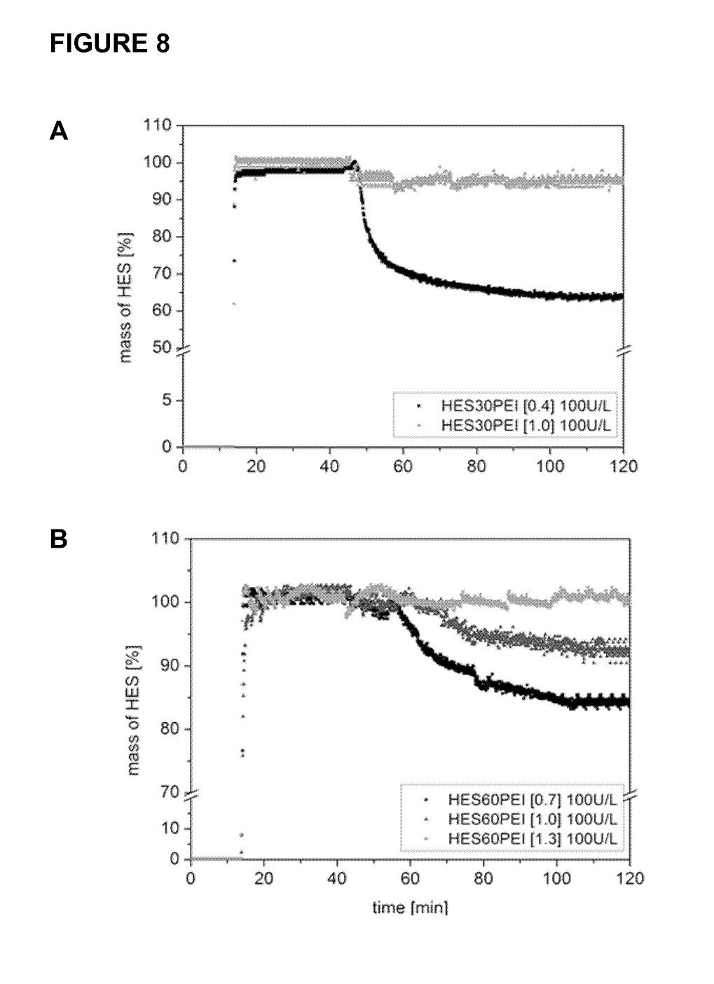

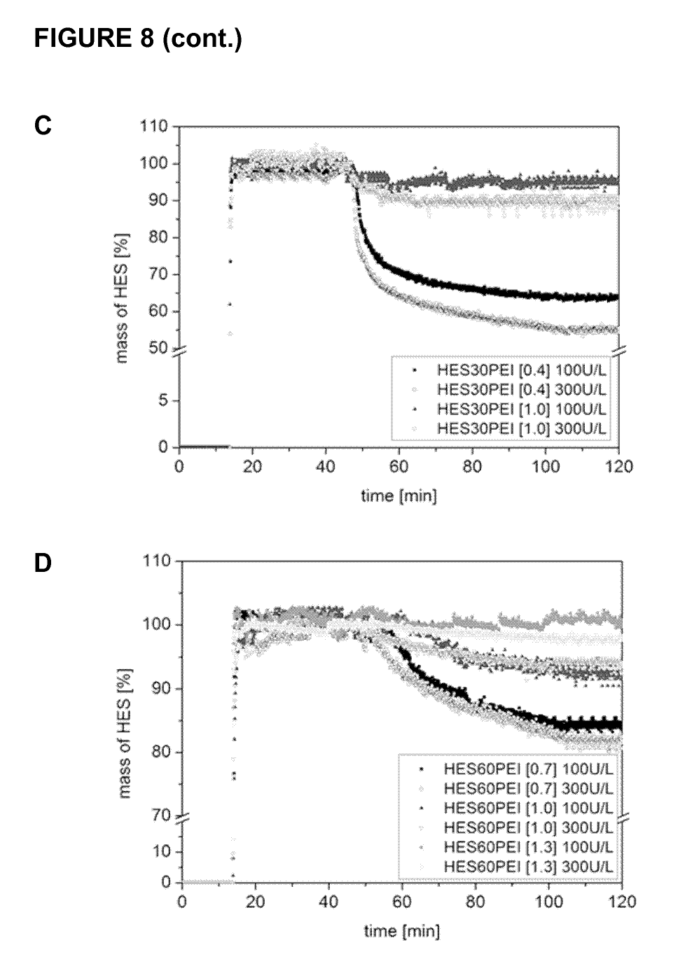

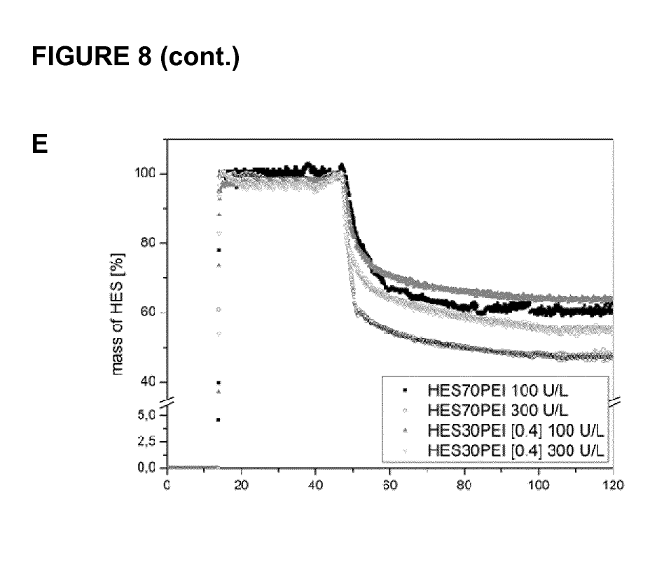

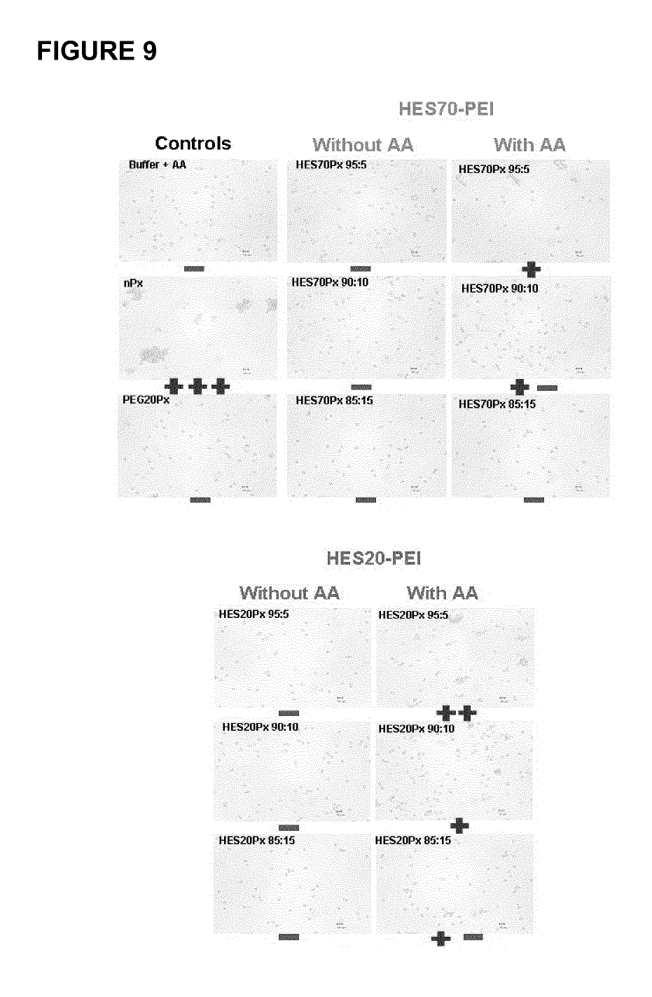

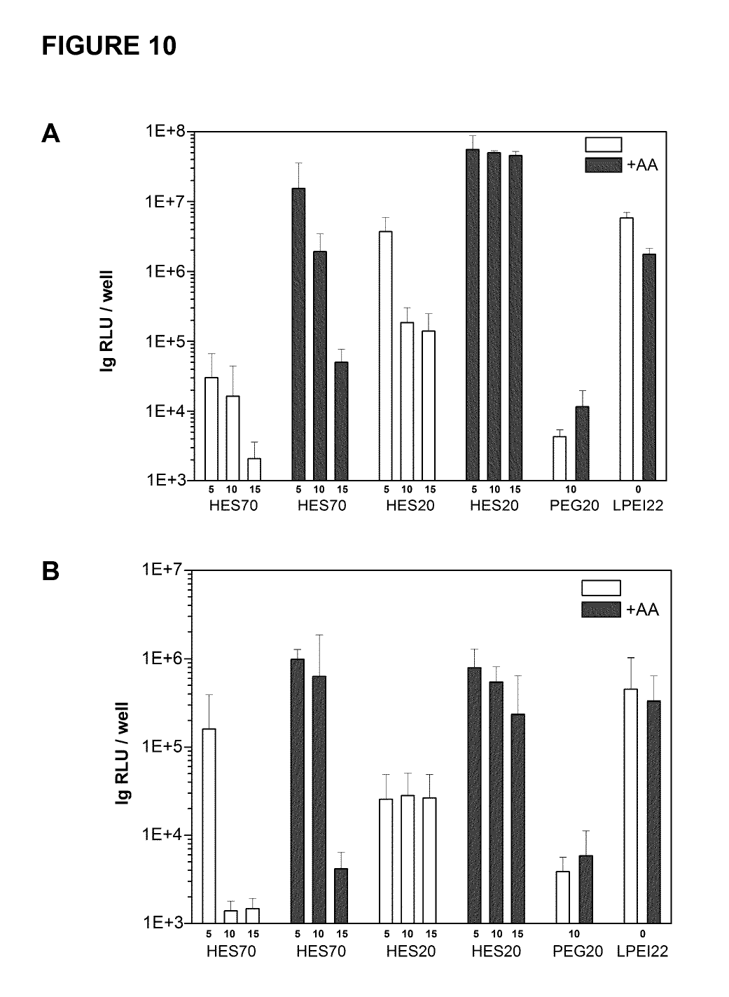

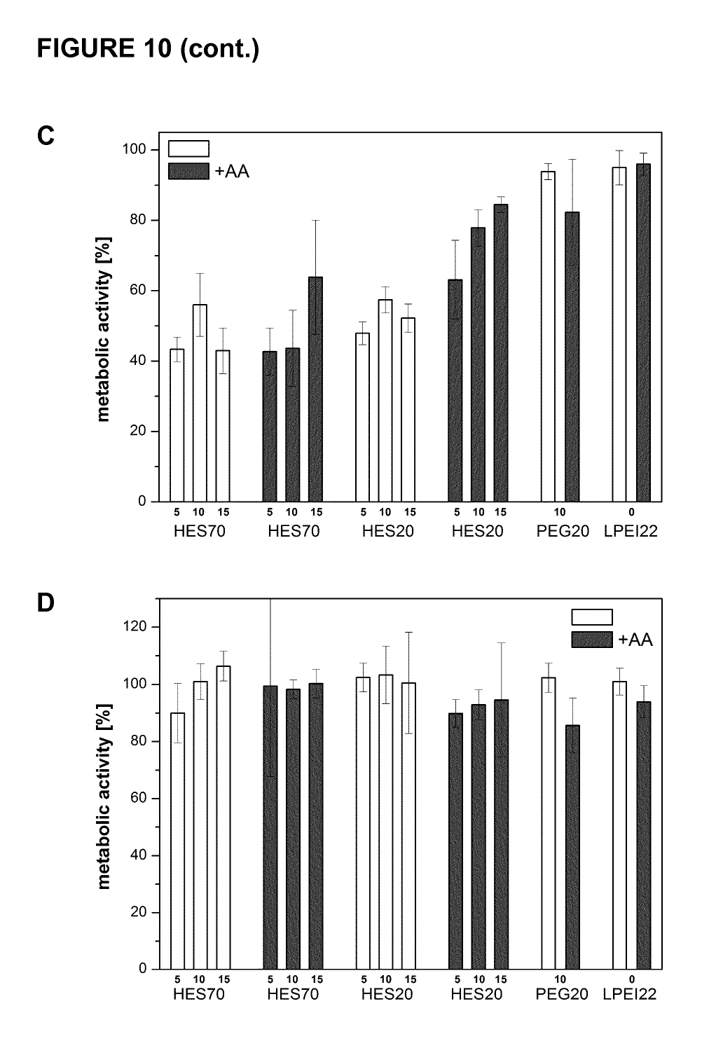

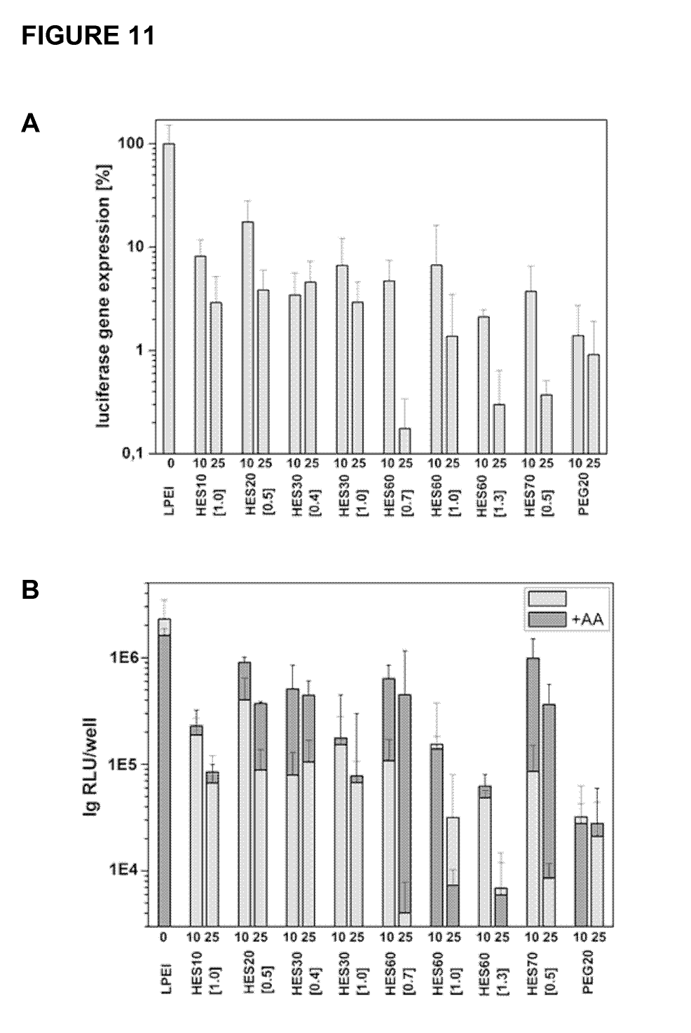

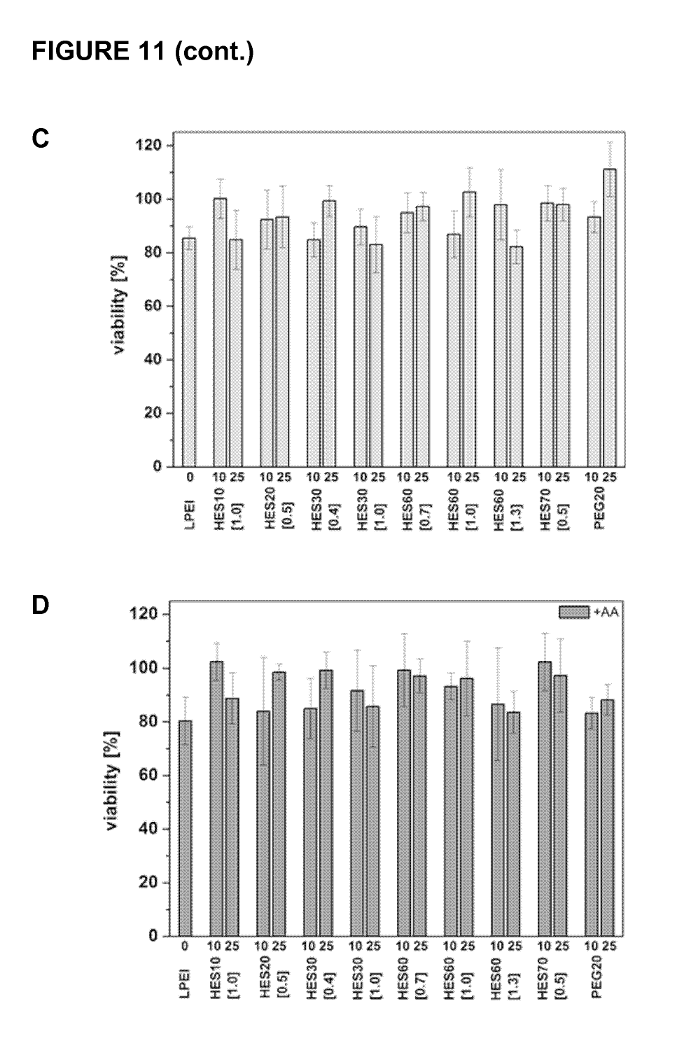

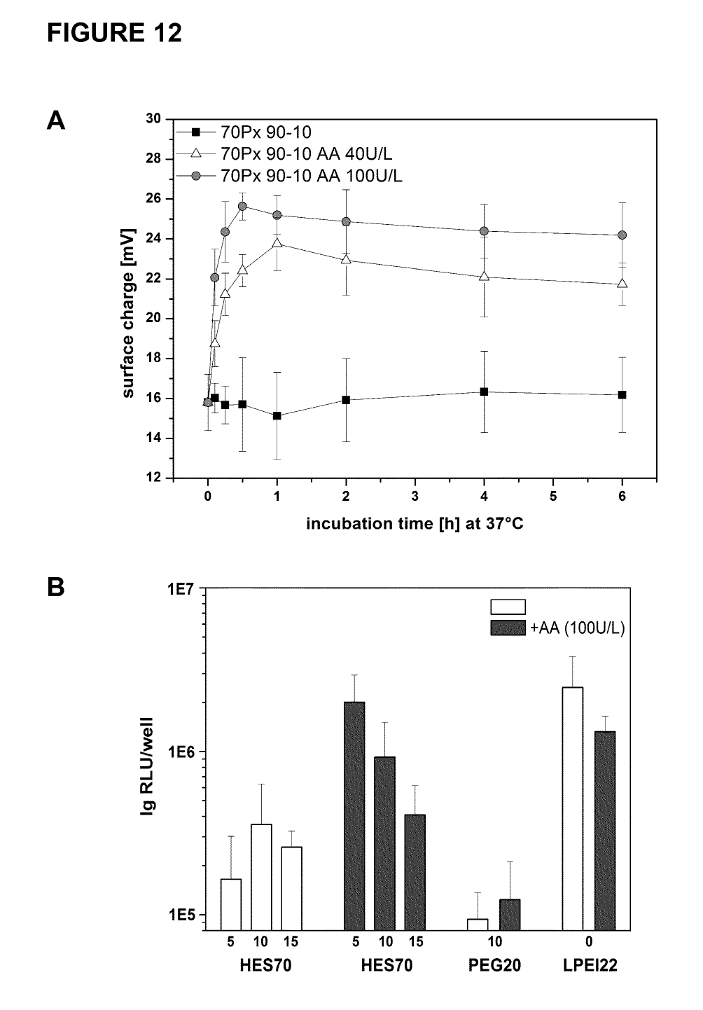

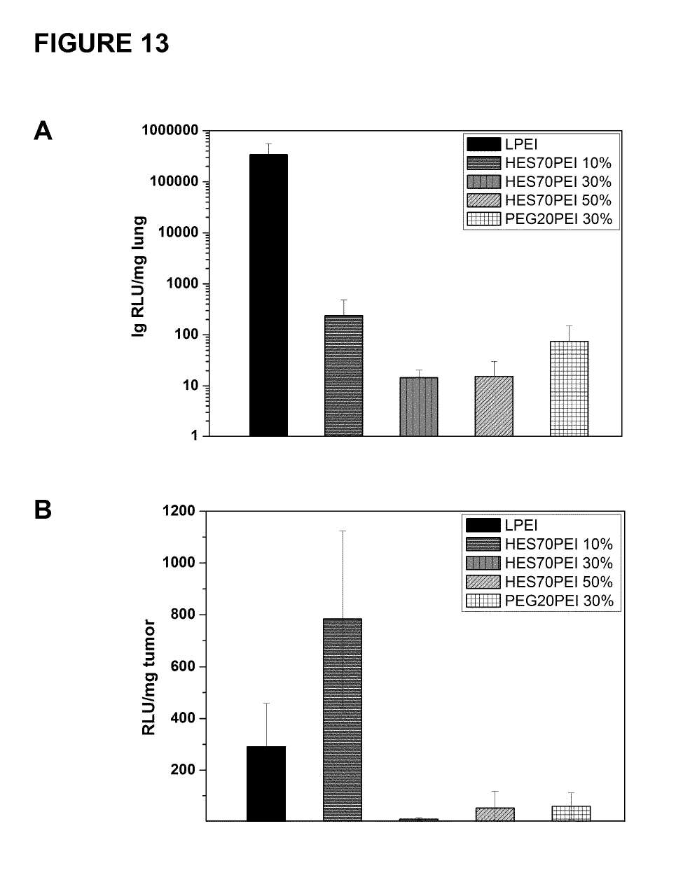

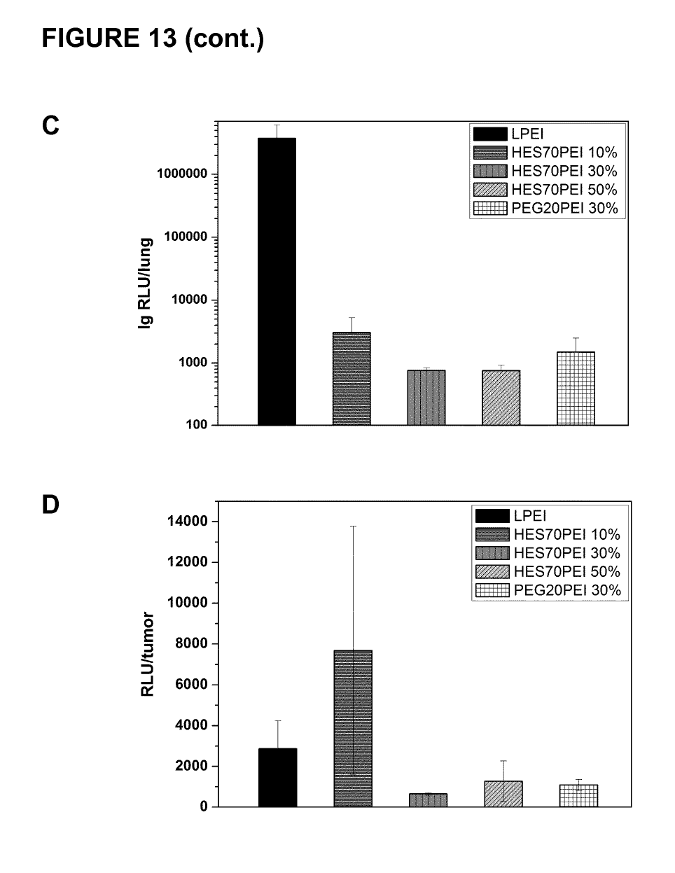

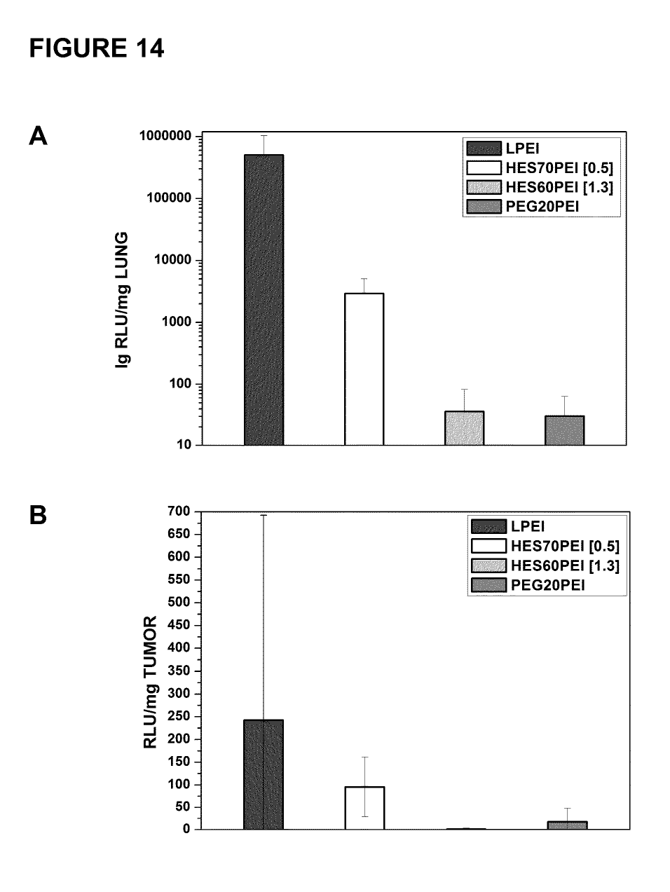

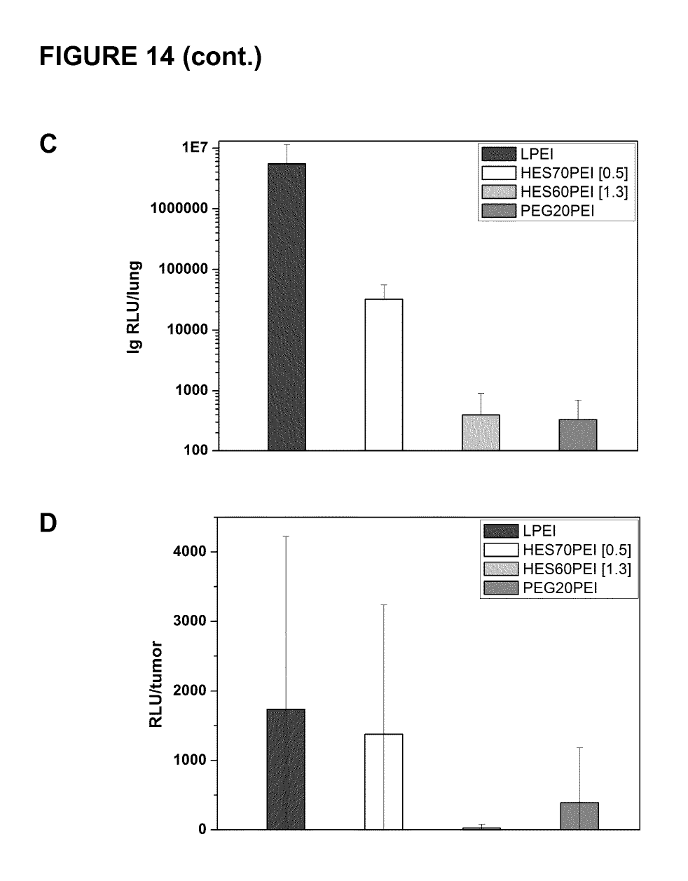

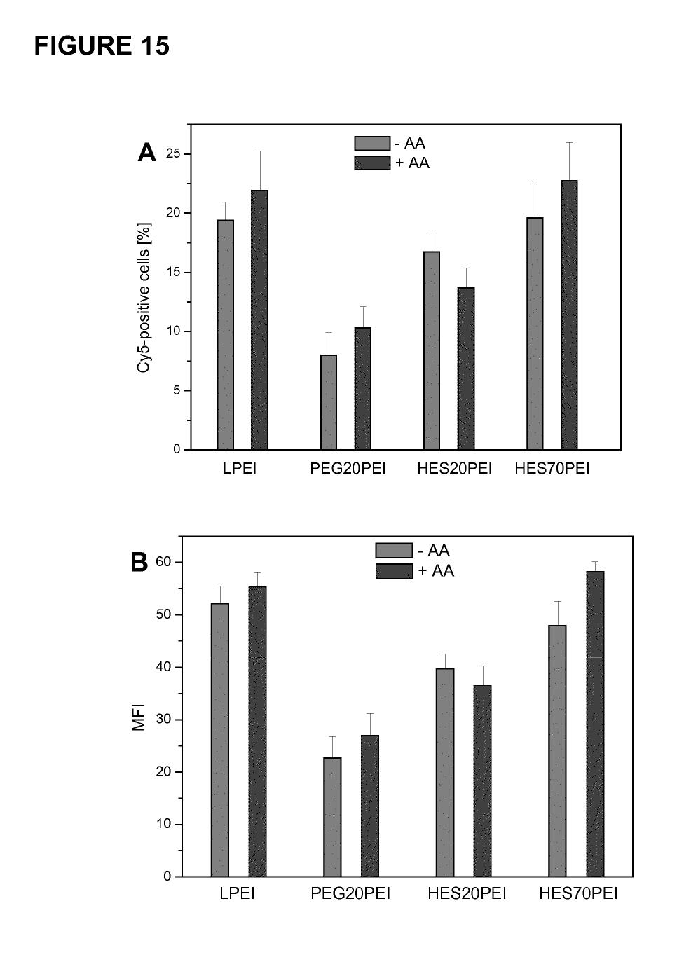

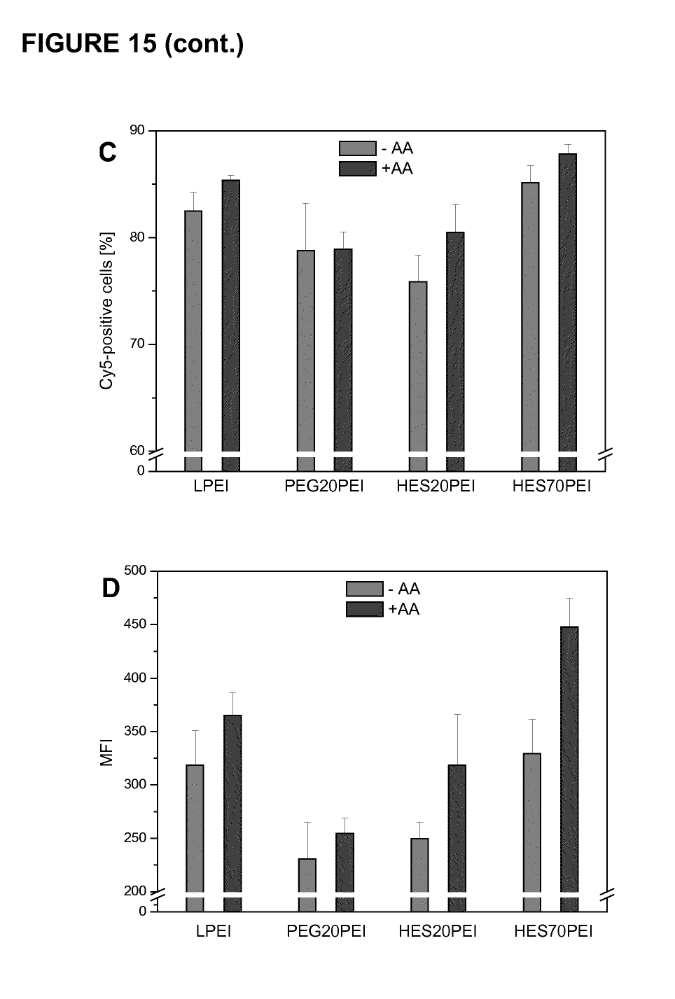

The ability of pancreatic α-amylase (AA) to cleave HES was evaluated under different reaction conditions (buffer composition, temperature, and pH). The concentration of AA was adjusted to an enzyme activity of 40 U/I. FIG. 1 depicts the degradation behavior of HES70 (A and C) and HES20 (B and D) as a function of time. Control samples lacking AA did not show any degradation. In the presence of enzyme, both HES70 and HES20 were initially degraded rapidly at 37° C., leveling-off after about 2 h, whereas degradation at 25° C. proceeded slower at the beginning but after 6 h resulted in a major fragmentation product of similar molar mass as that observed at 37° C. In total, HES70 lost approximately 50% of its initial molar mass after 6 h, while HES20 lost about 20%.

The pH had no apparent effect on degradation rate as can be seen when comparing the results obtained with HBG buffer at pH 7.1 and pH 6.0, respectively (FIG. 1E). However, degradation was significantly faster in PBS, pH 7.4 as compared to HBG, pH 6. This behavior is probably due to the fact that α-amylase is more active and stable in the presence of chloride ions (Caldwell, M. L. et al. (1953) J. Am. Chem. Soc. 75, 3132-3135).

### 2.3 Polycation Modification: Coupling of HES to PEI

HES-PEI copolymers were prepared by coupling HES20 and HES70 to the linear polyamine polyethylenimine (having an average molecular weight of 22 kDa; PEI22), respectively. The overall synthesis scheme is illustrated in FIG. 2. The coupling reaction was performed in PBS pH 7.4 using a large excess of HES (molar ratio 25:1) to ensure Schiff's base formation between the terminal aldehyde group of HES and the amine group in PEI. The HES moiety was coupled to PEI via an unstable aminol intermediate that immediately rearranged to an enamine function. Subsequently, the reducing agent sodium cyanoborohydride was added 2 h after start of the reaction to reduce the enamine to secondary (or tertiary) amine groups. Ion exchange chromatography was used to purify the conjugates, which were characterized using ¹H NMR, UV spectroscopy (copper assay), and size exclusion chromatography.

### 2.4 Characterization of Polymeric HES-PEI Conjugates

In order to determine the coupling efficiencies and the molar ratios of the resulting HES-PEI conjugates ¹H NMR measurements were performed for HES20-PEI22 and HES70-PEI22. FIG. 3 depicts the NMR spectra of both conjugates with assignment of corresponding peaks. The peaks between 5.3 ppm and 5.7 ppm relate to the proton at position C1 of the anhydro-glucose unit (AGU) of HES, and the peaks between 2.8 ppm and 3.1 ppm relate to the four protons of the ethylene group of PEI. The molar ratios of HES:PEI were determined as follows: HES20:PEI22=1.44:1, and HES70:PEI22=2.35:1 (see also Table 1).

In order to corroborate the results of ¹H NMR spectroscopy, a copper assay was performed for determining the amount of PEI in the HES20-PEI22 and HES70-PEI22 conjugates. The photometric copper-complex assay is based on the formation of a bluish complex of copper (II) ions with PEI that is detectable via spectroscopy at λₘₐₓ 285 nm (Ungaro, F. et al. (2003) J. Pharm. Biomed. Anal. 31, 143-149). A mixture of HES and PEI (non-conjugated) was used as a negative control and revealed no interference for HES. The results obtained were in excellent agreement with the NMR measurements (see Table 1 below).

SEC-MALS, a combination of size exclusion chromatography with multi angle light scattering is a common technique applied for the characterization of polymeric complexes/particles. The SEC chromatograms (FIG. 4) verified the respective coupling of HES20 and HES70 to the linear polyamine PEI22. As expected, the HES-PEI conjugates eluted earlier than mixtures of non-conjugated HES and PEI. Additionally, a low amount of unbound PEI22 appeared in the chromatograms.

TABLE 1
Mass ratios and molar ratios for HES20-PEI22 and HES70-PEI22
conjugates, as determined by ¹H-NMR and photometric
copper assay, respectively.

| | Amount of HES20 | | Amount of HES70 | |
|---|---|---|---|---|
| | Mass | Molar ratio | Mass | Molar ratio |
| | ratio [%] | HES20:PEI22 | ratio [%] | HES70:PEI22 |
| ¹H-NMR | 56.7 | 1.44:1 | 88.2 | 2.35:1 |
| Copper assay | 56.7 ± 8.8 | 1.44:1 | 88.3 ± 1.5 | 2.37:1 |

### 2.5 Biophysical Characterization of the Generated Polyplexes

The effect of different N/P ratios and various ratios of free PEI to HES-PEI on the formation of the polymeric complexes and their biophysical properties, particularly the particle size and the zeta potential as a measure of surface charge, was evaluated.

Polymeric complexes (“naked polyplexes”) were prepared by mixing PEI and pCMVLuc plasmid DNA (pDNA) (final DNA concentration of 20 μg/ml in HBG, pH 7.1) at N/P ratios of 3.6, 4.8, 6.0, 7.2, and 8.0. HESylated polymeric complexes were produced by partially replacing PEI with HES70- or HES20-modified PEI. These complexes were each generated with PEI:HES-PEI ratios of 95:5, 90:10, and 85:15, respectively. PEG20-PEI complexes as a control were prepared with a PEI:PEG-PEI ratio of 90:10. Particle size and zeta potential determinations of the various polyplexes were performed in HBG, pH 6.0 or pH 7.1 (FIG. 5).

By increasing the N/P ratio, the particle size of the polyplexes tended to decrease, but then leveled off at a size of approximately 70 nm at N/P ratios ≧6.0 (top and middle). At lower N/P ratios, the naked polyplexes tended to aggregate, apparently since there were not enough excess surface charges for particle stabilization. In contrast, the HES- and PEG-modifications imparted additional steric stabilization and prevented aggregation. It is worth noting that the HES70-PEI conjugate produced smaller particles at an N/P ratio of 3.6 as compared to both naked and PEG-PEI polyplexes. It is tempting to speculate that by using HES-PEI conjugates more stable polyplexes could be produced. This would, in turn, also result in reduced toxicity as low amounts of PEI were required. In addition, the zeta potential of the different polyplexes was determined as a function of increasing N/P ratios (bottom). From the results obtained, it is apparent that the water soluble polymers employed shielded the nanoparticles and thus reduced the zeta potential with the following order of efficacy: HES70>PEG20>HES20.

### Example 3

### Biochemical Characterization of Polymeric Complexes

### 3.1 Treatment of the Polymeric Complexes with α-Amylase

In order to establish an in vitro model for the enzymatically-catalyzed deshielding of the polymeric DNA complexes (i.e. the removal of the HES moiety), the effect of α-amylase (AA) on the zeta potential and size of the HES-decorated polyplexes was evaluated (FIG. 6). The stability of naked polyplexes (A) and HES70-PEI polyplexes (B) in HBG buffer at pH 7.1 or pH 6.0 was monitored over a period of 6 h after addition of AA.

The results show that the both types of polyplexes were not stable over six hours at pH 7.1 and physiological temperature, where the zeta potential decreased, while the particle size increased. In contrast the zeta potential and particle size of the polyplexes at pH 6.0 revealed a considerably higher stability, probably due to additional positive charges at this pH. Accordingly, further analyses were performed at pH 6.0.

The effect of AA on the stability of HES70-PEI polymeric complexes having different ratios of PEI to HES-PEI (95:5, 90:10, and 85:15) is shown in FIG. 7. At the lowest amount of HES70-PEI (5%), an increase in zeta potential could be observed, though not statistically significant. When using higher amounts of the conjugate (10% and 15%), after addition of AA, the zeta potential increased gradually before leveling off after about 1 h.

Furthermore, the addition of AA resulted in a (statistically significant) reduction of particle size of approximately 5-7 nm, which might be seen as another indication of enzymatic deshielding. After incubation of 4-6 h, the polyplexes treated with AA showed a re-increase in size. This might point to destabilization of the particles due to a reduced steric stabilization after removal of HES.

### 3.2 Quartz Crystal Microbalance with Dissipation (QCM-D) Experiments

QCM-D technology was used to study the kinetics and the extent of degradation of different HES-PEI polymers (cf. Table 2) in response to 100 U/I and 300 U/I α-amylase, respectively.

TABLE 2
HES-PEI conjugates and the amounts of HES coupled to PEI.
“HES” represents the average molar mass in kDa, and
“MS” the degree of molar substitution.

| | | Amount of HES | | Amount of HES | |
|---|---|---|---|---|---|
| | | (¹H NMR) | | (UV λ 285 nm) | |
| HES | | Mass ratio | Molar ratio | Mass ratio | Molar ratio |
| (kDa) | MS | [%] | HES:PEI | [%] | HES:PEI |
| 10 | 1.0 | 35.12 | 1.36:1 | 28.04 ± 2.29 | 0.98:1 |
| 20 | 0.5 | 56.70 | 1.64:1 | 56.70 ± 8.83 | 1.64:1 |
| 30 | 0.4 | 32.53 | 0.40:1 | 30.01 ± 3.56 | 0.36:1 |
| 30 | 0.4 | 74.74 | 2.47:1 | 72.83 ± 1.06 | 2.24:1 |
| 30 | 1.0 | 50.39 | 0.85:1 | 51.01 ± 3.44 | 0.87:1 |
| 60 | 0.7 | 79.97 | 1.67:1 | 79.62 ± 1.44 | 1.63:1 |
| 60 | 1.0 | 75.40 | 1.28:1 | 76.01 ± 0.71 | 1.32:1 |
| 60 | 1.3 | 79.97 | 1.67:1 | 77.17 ± 4.51 | 1.41:1 |
| 70 | 0.5 | 88.20 | 2.68:1 | 88.31 ± 1.49 | 2.71:1 |

In a first series of degradation experiments, HES-PEI conjugates were loaded onto the SiO₂-coated quartz crystal, followed by the supplementation of the enzyme. Adsorbed and desorbed mass was measured as changes in the frequency of the crystal, where the gradual biodegradation of HES by α-amylase was quantified as reduction of mass (using the Sauerbrey equation, overtone number 3).

As illustrated in FIGS. 8A and 8B, the molar substitution of HES has a considerable impact on the degradation profile of HES. The higher the degree of hydroxyethylation, the slower the cleavage of the α-1,4-glycosidic bonds of HES. HES30-PEI[0.4] showed rapid degradation at the beginning, followed by a phase of delayed loss of mass. The higher substituted HES30-PEI[1.0] copolymer was degraded very slowly. After 1 h treatment with 100 U/I α-amylase HES30-PEI[0.4] lost about 35% of its initial mass, and HES30-PEI[1.0] about 5-10%. On the other hand, HES60-PEIs were degraded in the following order with respect to the degree of molar substitution: 0.7>1.0>1.3 (loss of mass about 15%, 5%, and 0%, respectively).

Increasing the enzyme activity to 300 U/I resulted in a more distinctive degradation (faster cleavage and higher extent of degradation), especially in the case of lower hydroxyethylated HES types (see FIGS. 8C, 8D and 8E). HES70-PEI[0.5] showed a faster and stronger degradation as compared to HES30-PEI[0.4], indicating that—beside the degree of molar substitution of HES—the C2/C6 substitution pattern also has a strong impact on the extent and kinetics of HES degradation (see FIG. 8E). In general, the main hydroxyethylation of HES precedes at position C2 or C6 of the anhydroglucose unit (AGU). A predominant modification of position C2 at HES″ AGU affects the biodegradability at higher levels in comparison to substitutions of position C6 due to very strong steric hindrance of the cleavable α-1,4-glycosidic bonds (Yopshida, M. and Kishikawa, T. (1984) Starch-Stärke 36, 167-169).

Naked and PEGylated control particles were not degraded. The use of BSA (instead of enzyme) showed no degradation either confirming enzyme-specific degradation.

HES30-PEI[0.4] was subjected to a second administration of amylase solution with no additional degradation of HES. Testing the amylase activity over time using the Phadebas amylase test showed full maintenance of the amylase activity for at least 2 h. In conclusion, the degree of molar substitution of HES, its molar mass, the ratio of substitution at C2:C6 as well as the activity of the α-amylase appear to represent important parameters for the controlled shielding and deshielding of HES-decorated particles.

### 3.3 Erythrocyte Aggregation Assay

The shielding of HESylated polyplexes and the controlled amylase-induced deshielding were further tested using an erythrocyte aggregation assay. As can be seen in FIG. 9, the application of naked polyplexes caused considerable aggregation of the erythrocytes due to the electrostatic interactions. On the other hand, shielding of the polyplexes with HES70-PEI or HES20-PEI (with different ratios of PEI:HES-PEI) prevented the formation of such aggregates. The addition of AA triggered enzymatic deshielding, which led to development of small erythrocyte aggregates. No such effect could be seen when employing PEG-PEI polyplexes. In case of HES, the deshielding behavior was apparently dependent on the molar mass and amount of HES on the surface of polyplexes (cf. FIG. 7).

### 3.4 Cell Culture Experiments—Analysis of Transfection Efficiency

In vitro transfection experiments were performed in murine N2A neuroblastoma (FIG. 10A) and human HUH7 hepatoma cell lines (FIG. 10B) in the presence or absence of α-amylase in order to evaluate the effect of HES-shielding and enzymatic deshielding on transfection efficiencies. In both cell lines, transfection efficiencies for HES70-PEI polyplexes were similar to PEG-PEI polyplexes but significantly less efficient than for naked polyplexes.

In N2A cells, further evidence for the HES shielding effect could be observed by its dependence on the molar mass of the polymeric complexes and amount of HES present. For instance, HES20 shows higher transfection efficiency (and thus lower shielding) as compared to HES70. Transfection efficiency decreased with an increasing amount of HES (see FIG. 10A). For both HES70 and HES20, the addition of AA to the culture medium resulted in an increase in transfection efficiency by 2-3 orders of magnitude. Such an effect was not found for naked polyplexes or PEG-PEI polyplexes. This observation demonstrated the specificity of this effect for HES. Notably, HES20-PEI polyplexes showed a higher efficiency as compared to naked polyplexes. This phenomenon requires further investigations in order to unravel the underlying mechanisms.

HUH7 cells showed fairly similar transfection efficiencies as N2A cells (FIG. 10B). In general, when using HES20-coated polyplexes transfection was more efficient (i.e. lower shielding efficiency) as compared to the application of HES70. The addition of AA led to an enhancement of transfection efficiency by 1-3 orders of magnitude.

Metabolic activity of the transfected N2A (FIG. 10C) and HUH7 (FIG. 10D) cells was analyzed by means of a MTT assay (Sigma-Aldrich, Steinheim, Germany). Metabolic activity of transfected N2A cells was lower in case of HES-PEI particles than for PEG-PEI or PEI particles. No such behavior was observed in HUH7 cells. Generally, HUH7 cells showed a slower proliferation rate, less efficient transfection performance, and higher sensitivity to cytotoxicity as compared to N2A cells. Hence, HES-PEI presumably interfered with proliferation of N2A cells (but not with cell viability), and thus led to the apparent reduction in metabolic activity due to the smaller number of cells. This effect was not observed in HUH7 cells. The reason why reduced metabolic activity was observed in N2A cells but not in HUH7 cells needs to be further investigated.

In a second series of experiments, the effect of surface charge shielding and enzymatic particle deshielding on the luciferase gene expression was studied in N2A neuroblastoma cells cultivated in DMEM in the presence or absence of 100 U/I α-amylase (AA). Surface modification using HES resulted in an up to 3 orders of magnitude lowered transfection efficiency as compared to unshielded LPEI-polymeric complexes.

Best shielding effects, associated with low transfection efficiency, were obtained for high amounts of high molar mass HES in the polymeric shell, while more efficient transfection and lower shielding was observed for low amounts of low molecular weight HES. For instance, HES10-shielded polyplexes (10%) showed much higher transfection efficiency in comparison with 25% HES60-decoration (see FIG. 11A). After addition of AA, the HES coat was successively degraded and the particle activated. In case of HESylated polyplexes, enzymatic activation led to an increase of luciferase gene expression by 1-2 orders of magnitude, whereas no effect could be determined for naked particles, as well the particles shielded with non-degradable polymers, namely HES60-PEI[1.3] and PEG. The effect of AA was largest in case of HES70[0.5], and HES60[0.7], both having a high molar mass (showing effective initial shielding) and a relatively low degree of molar substitution (allowing effective deshielding by AA). The effect of AA on increasing in vitro transfection was also obvious to a lower extent in HES20[0.5], and HES30[0.4]. While these polymers have a low degree of molar substitution enabling the deshielding action of AA, they show a low initial shielding effect, thus attenuating the effect of deshielding.

These results are a clear indication for selective particle activation of HESylated nanoparticles using AA. De-HESylated transfection particles exhibited transfection levels almost equivalent to naked polyplexes (see FIG. 11B). The viability of N2A cells was not affected by the treatment with polymeric complexes, independent of the presence of AA (FIGS. 11C and 11D).

### 3.5 Effect of α-Amylase (AA) Activity

The effect of AA activity (40 U/I and 100 U/I) on the biophysical properties of the polyplexes as well as transfection efficiency was investigated. Amylase activity was determined using Phadebas® Amylase Test, for which a clinical serum activity in the range of 60-310 U/I has been reported (Bretaudiere, R. et al. (1981) Clin. Chem. 27, 806-815). Determining the zeta potential of the HES70-coated polyplexes (PEI:HES-PEI 90:10) incubated with the different amylase activities over 6 h showed that an increase the AA activity accelerates the increase in zeta potential, so that a plateau is reached after only 0.5 h rather than 1 h (FIG. 12A).

In addition, the higher amylase activity apparently increased the zeta potential to a higher level (though the difference in the plateau region is not statistically significant). The effect of amylase activity on the transfection efficiency of the HES70-coated polyplexes in N2A neuroblastoma cells was also investigated. The polyplexes treated with the higher amylase activity showed the same deshielding behavior, reaching transfection efficiencies similar to naked polyplexes (FIG. 12B), while it had no effect on the controls (PEG-coated polyplexes and naked polyplexes). These results show that the amylase activity has an effect on HES degradation and deshielding kinetics in vitro, but less influence on the extent of transfection.

### 3.6 Determining of the Amount of HESPEI Appropriate for In Vivo Applications

In vivo gene expression studies were performed in order to determine the amount of HES70-PEI (relative to PEI), which results in the lowest lung expression and the highest tumor expression rates, respectively. The composition of naked and variously modified LPEI-polymeric complexes employed is shown in Table 3.

TABLE 3
In vivo analysis for determining the amount of HES-PEI
(in relation to unmodified LPEI) in the polymeric complexes.

| | Amount of | Molar ratio [%] | Mass ratio [%] |
|---|---|---|---|
| | conjugate based | HES/PEG to total | HES/PEG to |
| | on LPEI [%] | polymer | total polymer |
| LPEI | — | — | — |
| HES70-PEI[0.5] | 10.00 | 21.14 | 42.90 |
| HES70-PEI[0.5] | 30.00 | 44.57 | 69.17 |
| HES70-PEI[0.5] | 50.00 | 57.26 | 78.89 |
| PEG20-PEI | 50.00 | 27.11 | 22.93 |

Naked LPEI-based DNA complexes strongly accumulated into the lung (see FIG. 13A; Zou, S. M. et al. (2000) J. Gene Med. 2, 128-134), while HES-containing particles—with 10%, 30% and 50% HES70-PEI[0.5]—resulted in a decrease in lung expression by 3 to 4 orders of magnitude (see FIG. 13A). Higher amounts of HES70-PEI (30% and 50%, ratio of conjugate to LPEI) and PEG20-PEI (30%) almost entirely blocked luciferase gene expression in the lung. Shielded nanoparticles circulate for longer time in the bloodstream, thus allowing for passive tumor targeting by the EPR effect (Greish, K. (2007) J. Drug Target. 15, 457-464). Biodegradable HES70-shielded particles with 10% conjugate resulted in a >2-fold increase in luciferase expression in the tumor (FIG. 13B) as compared to naked LPEI complexes. Increasing the amount of HES70-PEI to 30% and 50% amount of conjugate (based on LPEI) showed very low expression levels in the lungs, but a drastically reduced gene expression in the tumor due to an inefficient deshielding of the higher amount of polymer in the particle corona. PEGylated control complexes could reduce the lung expression by shielding, but showed very low tumor activity due to hindered cellular uptake and endosomal escape of the non-degradable PEG.

Excellent results were obtained for HES70-containing polymeric complexes with 10% HES70-PEI and 90% unmodified LPEI22. This particular complex reduced luciferase gene expression in the lungs by 3 orders of magnitude, while doubling the expression levels in the tumor as compared to the unmodified particles. It can be speculated that this finding is presumably due to prolonged circulation time by shielding, allowing the passive tumor targeting by the EPR effect in combination with extracellular particle activation by the controlled polymeric complex deshielding under the effect of serum amylase.

### 3.7 Determination of Luciferase Gene Expression after Systemic Application

Naked LPEI particles, degradable HES70-coated polymeric complexes as well as non-degradable HES60- and PEG20-coated polymeric complexes were used for investigating the effect of incorporation of degradable/not-cleavable hydrophilic polymers into the polyplex system. Based on the composition of the lead candidate HES70-coated polymeric complexes with 10% HES70PEI, the non-degradable HES60[1.3] and PEG20-containing polymeric complexes were prepared in the same molar ratio (as shown in Table 4).

TABLE 4
Composition of various LPEI-based transfection particles.
Unmodified and modified polymeric complexes were generated
at an N/P ratio 6.0. Modified particles had the same molar ratio
(21.14%, HES or PEG to total polymer), corresponding to a similar
mass ratio HES to total polymer in the case of HES70- and
HES60-polymeric complexes.

| | Amount of | Molar ratio [%] | Mass ratio [%] |
|---|---|---|---|
| | conjugate based | HES/PEG to total | HES/PEG to |
| | on LPEI [%] | polymer | total polymer |
| LPEI | — | — | — |
| HES70-PEI[0.5] | 10.00 | 21.14 | 42.90 |
| HES60-PEI[1.3] | 16.05 | 21.14 | 39.07 |
| PEG20-PEI | 21.61 | 21.14 | 17.67 |

In general, the systemic administration of naked LPEI-based gene delivery systems resulted in an uncontrolled nucleic acid delivery to the organs kidney, spleen, liver and lung. Highest gene expression levels can usually be observed in the lung (Goula, D et al. (1998) Gene Therapy 5, 1291-1293). The incorporation of HES and PEG in the polymeric complexes reduced the gene expression in the lungs by 2-4 orders of magnitude (FIG. 14A). However, shielding of the polymeric complexes with the non-degradable HES60[1.3] or PEG20 resulted in an almost entire loss of gene expression in the tumor tissue. Meanwhile, the use of the biodegradable HES70[0.5] maintained the transfection efficiency in the tumor.

In addition to the mean values of gene expression, the number of animals with tumor expression is shown in Table 5. Unmodified and HES70-decorated polymeric complexes showed high levels of gene expression in 5/5 and 4/5 mice, respectively, while non-reversible shielded particles led to low transfection levels in only 1/5 or 2/5 mice.

TABLE 5
Gene expression data in tumor tissue (RLU/mg organ). 5/5 and 4/5 mice showed
tumor expression for the LPEI and HES70-coated particles, respectively. 1/5 and 2/5
mice showed tumor expression for the complexes coated with non-degradable
polymers, HES60-PEI[1.3] and PEG, respectively. Gene expression levels are high-
lighted in shades of green: High level (dark), medium level (medium), low level (light).

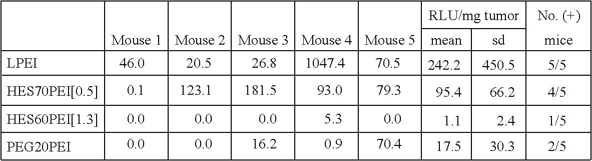

### 3.7 Binding and Uptake Experiments Using Flow Cytometry

The in vitro binding and uptake capacity of the HES-coated polyplexes was investigated by using flow cytometry. Binding was performed at 4° C., where only non-specific adsorption to the cell-surface takes place, while the energy-dependent uptake is inhibited. The results of FIGS. 15A and 15B show that PEG20 and HES20 were effective in inhibiting binding of the HES-coated polyplexes (compared to naked polyplexes used as control), as it is illustrated in the percentage of cells associated with labelled-DNA as well as the mean fluorescence intensity (“MFI”, an indicator for the average amount of labeled DNA per cell). These data demonstrate the effective shielding of PEG20 and HES20, and their ability to reduce non-specific adsorption. The apparent ineffectiveness of HES70 in preventing non-specific adsorption contradicts with results from transfection efficiency and erythrocyte aggregation assay, and is probably difficult to interprete at this stage. Finally, the addition of AA does not have a significant effect on binding, probably due to the low activity of AA at 4° C.

Results of the uptake analysis at 37° C. (FIGS. 15C and 15D) show that the percentage of Cy5 positive cells is much higher than at 4° C., but does not differ significantly between the different types of polyplexes before or after addition of AA. The percentage of Cy5-positive cells is approximately 75-85%. On the other hand, MFI decreases for PEG20- and HES20-coated polyplexes, which is in accordance with the binding experiments. Furthermore, the addition of AA results in a significant increase in MFI by 28% and 36% for the HES20- and HES70-coated polyplexes, respectively, without having marked effect on the controls.

The above results are in agreement with those reported with Nie et al. (Nie, Y. et al. (2011) Biomaterials 32, 858-869) in connection with an analysis of PEGylation on the binding and uptake of cationic lipopolyplexes for DNA transfection. Although the addition of AA does not affect the percentage of cells which actively phagocytose the polyplexes, it can significantly increase the amount of DNA being delivered per cell, which is an indication for effective degradation of the polymer shell. Results of the flow cytometry analysis further demonstrate that HES-coated polyplexes can be effectively deshielded by amylase, resulting in an increase in the amount of DNA delivered per cell. This deshielding is also expected to reduce the interference with the endoplasmic escape and increase transfection efficiency as already observed with the luciferase transfection experiments.

### 3.8 Conclusions

The results obtained provide evidence that hydroxyethyl starch (HES) can be successfully applied for the shielding and controlled (i.e. enzyme-catalyzed) deshielding of polymeric DNA complexes.

HES70-PEI and HES20-PEI conjugates were used to form stable polymeric complexes with plasmid DNA. Biophysical characterization of such polymeric complexes having different N/P ratios revealed that both their hydrodynamic diameters and surface charges were similar to corresponding PEGylated conjugates. The effect of α-amylase on the zeta potential of HESylated polymeric complexes was analyzed in vitro, showing a gradual increase in the surface charge of the nanoparticles up to 1 h, indicating effective enzymatic deshielding. Furthermore, α-amylase treatment of HESylated polymeric complexes also caused erythrocyte aggregation, while no such effect occurred in the absence of enzyme. In vitro transfection experiments with two different cell lines revealed that the presence of α-amylase specifically increased the transfection efficiency of HES-coated particles by 2-3 orders of magnitude. No effect was found with PEG-coated or uncoated particles.

Hence, HES-PEI conjugates represent a suitable molecular tool for the controlled shielding/deshielding of polymeric DMA complexes for gene delivery. The option to specifically manipulate the rate and extent of HES biodegradation by varying its molecular weight and degree of molar substitution (i.e. hydroxylation) offers a promising approach for engineering “customized” polymeric complexes having a purpose-specific degradation profile for the controlled intracellular delivery of nucleic acids.

The in vivo results confirmed that the shielding and deshielding concept is a suitable and promising approach for delivering nucleic acids into target cells, thereby maintaining particle stability in the bloodstream as well as transfection efficiency by enzymatic particle activation. Although the degradable HES70- and non-degradable HES60-coated polymeric complexes have the same molar ratios of HES, they show different expression levels in the tumor, indicating that indeed the degradation of HES results an increased transfection in the tumor. The data obtained reveal that the degree of molar substitution, total amount and the molecular weight of HES are important factors for controlling the biodegradation of HESylated systems in vitro and in vivo.

The present invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modifications and variations of the inventions embodied therein may be resorted to by those skilled in the art, and that such modifications and variations are considered to be within the scope of this invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. Method for the controlled intracellular delivery of nucleic acid molecules into one or more target cells, the method comprising:
(a) providing a shielded polymeric complex formed between one or more nucleic acid molecules to be delivered and one or more cationic carrier molecules, wherein at least a part of the one or more carrier molecules in the polymeric complex are covalently attached to hydroxyalkyl starch, and wherein the hydroxyalkyl starch is shielding the polymeric complex;
(b) allowing the shielded polymeric complex to get into contact with the one or more target cells;
(c) deshielding the polymeric complex by removing the hydroxyalkyl starch; and
(d) allowing the deshielded polymeric complex to internalize into the one or more target cells.

2. The method of claim 1, wherein the hydroxyalkyl starch is hydroxyethyl starch.

3. The method of claim 2, wherein the hydroxyethyl starch has:
(i) an average molecular weight in the range between 2 kDa and 300 kDa, and particularly in the range between 10 kDa and 200 kDa; and
(ii) an average number of hydroxylethyl groups per glucose unit in the range between 0.1 and 2.0, and particularly in the range between 0.1 and 1.0.

4. The method of claim 1, wherein in the shielded polymeric complex the molar ratio between free and hydroxyalkyl starch-modified carrier molecules is in the range between 1:99 and 99:1, and particularly in the range between 5:95 and 95:5.

5. The method of claim 1, wherein the hydroxyalkyl starch is removed enzymatically by exposing the shielded polymeric complex to amylase, and particularly to α-amylase.

6. The method of claim 5, wherein the amylase is exogenously added to the one or more target cells.

7. The method of claim 5, wherein the extent of modification with hydroxyalkyl starch is indicative of the amount of amylase required for substantially removing the hydroxyalkyl starch.

8. The method of claim 1, wherein the shielded polymeric complex further comprises one or more targeting molecules for the specific delivery of the one or more nucleic acid molecules to the one or more target cells.

9. The method of claim 1, wherein the one or more carrier molecules are selected from the group consisting of cationic lipids, cationic cholesterol-complexes, cationic peptides, in particular poly-arginines and poly-lysines, polyalkylenimines, in particular polyethylenimine, protamines, and combinations thereof.

10. The method of claim 1 wherein the one or more nucleic acid molecules are selected from the group of RNA molecules, in particular siRNA molecules, miRNA molecules, and shRNA molecules, and precursor molecules thereof, and DNA molecules.

11. The method of claim 1, wherein the one or more target cells are tumor cells.

12. The method of claim 11, wherein the tumor cells are amylase-producing tumor cells.

13. Use of a method as defined in claim 1 for the delivery of one or more therapeutically active nucleic acid molecules into one or more target cells.

14. The use of claim 13, wherein the one or more therapeutically active nucleic acid molecules are applied for the prevention and/or treatment of a condition selected from the group consisting of cancer, immune diseases, cardiovascular diseases, neuronal diseases, infections, and inflammatory diseases.

15. Pharmaceutical composition, comprising a shielded polymeric complex as defined in claim 1, and optionally further comprising amylase, for use in the prevention and/or treatment of a condition selected from the group consisting of cancer, immune diseases, cardiovascular diseases, neuronal diseases, infections, and inflammatory diseases.
